SEQUENCE LISTING (5 sequences)

SEQ ID NO: 1 - protein, 720 aa, Artificial Sequence
  source: /note=“Description of Artificial Sequence Synthetic polypeptide”
EVQLVESGGGLVQPGRSLRLSCAASGFTFDDYAMHWVRQAPGKGLEWVSGISWDSGSVGYADSVKGRFTISRDNAKNSLYLQMNSLRAEDTALYYCARDLGYNQWWEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLQSGGGLVQPGGSLRLSCAASGFDFSSYPMHWVRQAPGKGLEWVGSISSSGGATPYADSVKGRFTISRDNSKNTLYLQMNSLRPEDTAVYYCAKDFYTILTGNAFDMWGQGTSVTVSSASTGGGGSGGGGSGGGGSGGGGSDIQMTQSPSSLSASLGDRVTITCRASQGISSYLAWYQQKPGKAPKLLIYASSTRQSGVPSRFSGSGSGTDFTLTISSLQPEDSGTYYCQQYWAFPLTFGGGTKVEIKRT

SEQ ID NO: 2 - protein, 214 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSRDTPGNKWVFGGGTKVTVIGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 3 - protein, 720 aa, Artificial Sequence
  source: /note=“Description of Artificial Sequence Synthetic polypeptide”
EVQLLQSGGGLVQPGGSLRLSCAASGFMFSRYPMHWVRQAPGKGLEWVGSISGSGGATPYADSVKGRFTISRDNSKNTLYLQMNSLRAEDTAVYYCAKDFYQILTGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGGGLVQPGGSLRLSCAASGFTFDDYAMHWVRQAPGKGLEWVAGISWDSGSTGYADSVKGRFTISRDNAKNSLYLQMNSLRAEDTALYYCARDLGAYQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSPGNQWVFGGGTKVTVLG

SEQ ID NO: 4 - protein, 214 aa, Artificial Sequence
  source: /note=“Description of Artificial Sequence Synthetic polypeptide”
DIQMTQSPSSLSASLGDRVTITCRASQGISSYLAWYQQKPGKAPKLLIYAKSTLQSGVPSRFSGSGSGTDFTLTISSLQPEDSATYYCQQYWTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 720 aa, Artificial Sequence
  source: /note=“Description of Artificial Sequence Synthetic polypeptide”
EVQLLQSGGGLVQPGGSLRLSCAASGFMFSRYPMHWVRQAPGKGLEWVGSISGSGGATPYADSVKGRFTISRDNSKNTLYLQMNSLRAEDTAVYYCAKDFYQILTGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGGGLVQPGGSLRLSCAASGFTFDDYAMHWVRQAPGKGLEWVAGISWNSGSIGYADSVKGRFTISRDNAKNSLYLQMNSLRPEDTAVYYCARDLGYNQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLG

(19) United States
(12) Patent Application Publication
(10) Pub. No.: US 2017/0096492 A1
(43) Pub. Date: Apr. 6, 2017

(54) DOSAGE AND ADMINISTRATION OF ANTI-IGF-1R, ANTI-ErbB3 BISPECIFIC ANTIBODIES, USES THEREOF AND METHODS OF TREATMENT THEREWITH

(71) Applicant: Merrimack Pharmaceuticals, Inc., Cambridge, MA (US)

(72) Inventors: Sharlene ADAMS, Waltham, MA (US); Jason BAUM, Needham, MA (US); Michael CURLEY, Boston, MA (US); Alexey Alexandrovich LUGOVSKOY, Woburn, MA (US)

(21) Appl. No.: 15/241,626

(22) Filed: Aug. 19, 2016

Related U.S. Application Data
(63) Continuation of application No. PCT/US2015/016672, filed on Feb. 19, 2015.
(60) Provisional application No. 62/005,333, filed on May 30, 2014.
(60) Provisional application No. 62/103,963, filed on Jan. 15, 2015.
(60) Provisional application No. 62/078,203, filed on Nov. 11, 2014.
(60) Provisional application No. 62/047,487, filed on Sep. 8, 2014.
(60) Provisional application No. 61/942,472, filed on Feb. 20, 2014.

(51) Int. Cl. C07K 16/32; A61K 31/337; A61K 47/42; A61K 31/7068; A61K 9/00; C07K 16/30; C07K 16/28; A61K 39/395

(52) U.S. Cl. CPC C07K 16/32; C07K 16/2863; A61K 31/337; A61K 39/39558; A61K 31/7068; A61K 9/0019; C07K 16/303; A61K 47/42; C07K 2317/31; C07K 2317/565; C07K 2317/35; A61K 2039/505

(57) ABSTRACT

Provided herein are compositions comprising anti-IGF-1R, anti-ErbB3 bispecific antibodies alone or in combination with other anti-cancer agents. Also provided are methods of treating a subject having cancer and methods for determining whether a patient with cancer is likely to respond to the compositions described herein.

### REFERENCE TO RELATED APPLICATIONS

This application is a continuation of International Application No. PCT/US2015/016672, filed Feb. 19, 2015, which claims priority to U.S. Provisional Application Ser. No. 62/103,963, filed Jan. 15, 2015, U.S. Provisional Application Ser. No. 62/078,203, filed Nov. 11, 2014, U.S. Provisional Application Ser. No. 62/047,487, filed Sep. 8, 2014, U.S. Provisional Application Ser. No. 62/005,333, filed May 30, 2014, and U.S. Provisional Application Ser. No. 61/942,472, filed Feb. 20, 2014. The contents of the aforementioned applications are hereby incorporated by reference.

### SEQUENCE LISTING

The instant application contains a Sequence Listing which has been submitted via EFS-Web and is hereby incorporated by reference in its entirety. Said ASCII copy, created on Aug. 19, 2016, is named MMJ_065PCCN_Sequence_Listing.txt and is 23,449 bytes in size.

### FIELD

Provided are methods of treating patient with cancer with targeted therapies alone or in combination with chemotherapies. Additionally, methods of determining whether the patient is likely to respond to a treatment with the aforementioned combinations are described.

### BACKGROUND

Cancer therapy treatment has advanced with the use of targeted agents that have significantly increased the utility of traditional chemotherapies as part of combination regimens. Most of the successes have been observed in those cancer subtypes in which a specific oncogenic protein is mutated, such as EGF receptor (EGFR), BRAF, or ALK, or the expression is amplified, such as ErbB2 in breast and gastric cancer. However, many patients never respond to these combination regimens or become refractory, suggesting the existence of uncharacterized tumor survival mechanisms. Although inhibition of IGF-1R was expected to eliminate a key resistance mechanism to anticancer therapies, clinical results to date have been disappointing. It has previously been established that adaptive v-erb-b2 erythroblastic leukemia viral oncogene homolog 3 (ErbB3) signaling activated by its ligand heregulin is a key factor limiting the utility of anti-IGF-1R agents. A series of biomolecules have been invented that co-inhibit IGF-1R and ErbB3, including a bispecific tetravalent antibody, MM-141. MM-141 is a polyvalent bispecific antibody (PBA) that co-blocks IGF-1 and heregulin-induced signaling and induces degradation of receptor complexes containing IGF-1R and ErbB3, including their respective heterodimers with insulin receptor and with ErbB2. MM-141 is disclosed in U.S. Pat. No. 8,476,409, which also discloses a number of other novel PBAs that, like MM-141, bind specifically to human IGF-1R and to human ErbB3 and are potent inhibitors of tumor cell proliferation and of signal transduction through their actions on either or (typically, as for MM-141) both of IGF-1R and ErbB3. The invention of such co-inhibitory biomolecules has resulted in a need for new approaches to combination therapies for cancer. The present invention addresses these needs and provides other benefits.

### SUMMARY

Provided herein are compositions comprising, and methods for use of PBAs. It has now been discovered that co-administration of such a PBA (e.g., MM-141, as described below) with one or more additional anti-cancer agents, such as everolimus, capecitabine, a taxane, or XL147, exhibits therapeutic synergy.

Accordingly, provided are methods for the treatment of a cancer in a human patient (a “subject”) wherein the methods comprise administering to the subject a therapeutically effective amount of an IGF-1R and ErbB3 co-inhibitor biomolecule.

In certain embodiments the IGF-1R and ErbB3 co-inhibitor biomolecule is co-administered with a phosphatidylinositide 3-kinase (PI3K) inhibitor. The biomolecule and the PI3K inhibitor may be in a single formulation or unit dosage form or the PI3K inhibitor and the biomolecule are each administered in a separate formulation or unit dosage form, or the PI3K inhibitor is administered orally, and the biomolecule is administered intravenously, or either or both of the PI3K inhibitor and the biomolecule are administered simultaneously or sequentially. In some embodiments the PI3K inhibitor is administered prior to the administration of the biomolecule. In others the PI3K inhibitor is administered orally, and biomolecule is administered intravenously.

In other embodiments, the patient is concurrently treated with the biomolecule and an anti-estrogen therapeutic agent and optionally with a PI3K inhibitor. The anti-estrogen therapeutic agent may be, e.g., exemestane, letrozole, anastrozole, fulvestrant or tamoxifen.

In any of the preceding embodiments, the biomolecule may be administered at a dosage of 20 mg/kg weekly or 40 mg/kg bi-weekly, or may be administered at a fixed dose of 2.8 g. In any of the preceding embodiments, 1) the biomolecule is MM-141 as described (as “P4-G1-M1.3”) in U.S. Pat. No. 8,476,409, and 2) the PI3K inhibitor is GSK2636771 (CAS#: 1372540-25-4) or TGX-221 (CAS#: 663619-89-4).

In any of the preceding embodiments, the cancer is sarcoma (e.g. Ewing's sarcoma, rhabdomyosarcoma, osteosarcoma, myelosarcoma, chondrosarcoma, liposarcoma, leiomyosarcoma, soft tissue sarcoma), lung cancer (e.g. non-small cell lung cancer and small cell lung cancer), bronchus, prostate, breast, pancreas, gastrointestinal cancer, colon, rectum, colon carcinoma, colorectal adenoma, thyroid, liver, intrahepatic bile duct, hepatocellular, adrenal gland, stomach, gastric, glioma (e.g., adult, childhood brain stem, childhood cerebral astrocytoma, childhood visual pathway and hypothalamic), glioblastoma, endometrial, melanoma, kidney, renal pelvis, urinary bladder, uterine corpus, uterine cervix, vagina, ovary (e.g., high-grade serous ovarian cancer), multiple myeloma, esophagus, brain (e.g., brain stem glioma, cerebellar astrocytoma, cerebral astrocytoma/malignant glioma, ependymoma, meduloblastoma, supratentorial primitive neuroectodermal tumors, visual pathway and hypothalamic glioma), lip and oral cavity and pharynx, larynx, small intestine, melanoma, villous colon adenoma, a neoplasia, a neoplasia of epithelial character, lymphomas (e.g., AIDS-related, Burkitt's, cutaneous T-cell, Hodgkin, non-Hodgkin, and primary central nervous system), a mammary carcinoma, basal cell carcinoma, squamous cell carcinoma, actinic keratosis, tumor diseases, including solid tumors, a tumor of the neck or head, polycythemia vera, essential thrombocythemia, myelofibrosis with myeloid metaplasia, Waldenstrom's macroglobulinemia, adrenocortical carcinoma, AIDS-related cancers, childhood cerebellar astrocytoma, childhood cerebellar astrocytoma, basal cell carcinoma, extrahepatic bile duct cancer, malignant fibrous histiocytoma bone cancer, bronchial adenomas/carcinoids, carcinoid tumor, gastrointestinal carcinoid tumor, primary central nervous system, cerebellar astrocytoma, childhood cancers, ependymoma, extracranial germ cell tumor, extragonadal germ cell tumor, extrahepatic bile duct cancer, intraocular melanoma eye cancer, retinoblastoma eye cancer, gallbladder cancer, gastrointestinal carcinoid tumor, germ cell tumors (e.g., extracranial, extragonadal, and ovarian), gestational trophoblastic tumor, hepatocellular cancer, hypopharyngeal cancer, hypothalamic and visual pathway glioma, islet cell carcinoma (endocrine pancreas), laryngeal cancer, malignant fibrous histiocytoma of bone/osteosarcoma, meduloblastoma, mesothelioma, metastatic squamous neck cancer with occult primary, multiple endocrine neoplasia syndrome, multiple myeloma/plasma cell neoplasm, mycosis fungoides, nasal cavity and paranasal sinus cancer, nasopharyngeal cancer, neuroblastoma, oral cancer, oropharyngeal cancer, ovarian epithelial cancer, ovarian germ cell tumor, ovarian low malignant potential tumor, islet cell pancreatic cancer, parathyroid cancer, pheochromocytoma, pineoblastoma, pituitary tumor, pleuropulmonary blastoma, ureter transitional cell cancer, retinoblastoma, rhabdomyosarcoma, salivary gland cancer, Sézary syndrome, non-melanoma skin cancer, Merkel cell carcinoma, squamous cell carcinoma, testicular cancer, thymoma, gestational trophoblastic tumor, and Wilms' tumor. The cancer may be a primary tumor; the tumor may be a metastatic tumor. The cancer may be pancreatic cancer, ovarian cancer (e.g., high-grade serous ovarian cancer, platinum resistant ovarian cancer, or high-grade serous platinum resistant ovarian cancer), sorafenib-naive or sorafenib-refractory hepatocellular carcinoma, parathyroid cancer, sarcoma, lung cancer or breast cancer. The cancer may be a KRAS mutant cancer (e.g., a KRAS mutant pancreatic cancer).

Treatment according to the present disclosure in any of its embodiments may be carried out by administering an effective amount of a bispecific anti-IGF-1R and anti-ErbB3 antibody to the patient, where the patient is given a single loading dose of at least 10 mg/kg of the bispecific antibody followed administration of one or more maintenance doses given at intervals. The intervals between doses are intervals of at least three days. In some embodiments, the intervals are every fourteen days or every twenty-one days.

The doses administered may range from 1 mg/kg to 60 mg/kg of the bispecific antibody. In some embodiments, the loading dose is greater than the maintenance dose. The loading dose may from 12 mg/kg to 20 mg/kg, from 20 mg/kg to 40 mg/kg, or from 40 mg/kg to 60 mg/kg. In some embodiments the loading dose is about 12 mg/kg, 20 mg/kg, 40 mg/kg, or 60 mg/kg. In other embodiments the maintenance dose is about 6 mg/kg, 12 mg/kg, 20 mg/kg, 30 mg/kg, 40 mg/kg, 50 mg/kg or 60 mg/kg.

In other embodiments, a fixed dose of the bispecific anti-IGF-1R and anti-ErbB3 antibody is administered, rather than a body-mass-based dose. In one embodiment, a dose of 2.8 grams is administered to the patient every two weeks (Q2W). In other embodiments, a dose of 2.24 grams Q2W, 1.96 grams Q2W, 1.4 grams Q1W, 1.4 grams Q1W×3 with 1W off, 40 mg/kg Q2W, or 20 mg/kg Q1W is administered.

In some embodiments the patient has a pancreatic cancer, renal cell carcinoma, Ewing's sarcoma, non-small cell lung cancer, gastrointestinal neuroendocrine cancer, estrogen receptor-positive locally advanced or metastatic cancer, ovarian cancer (e.g., high-grade serous ovarian cancer), colorectal cancer, endometrial cancer, or glioblastoma. In one embodiment, the patient has a cancer that is refractory to one or more anti-cancer agents, e.g., gemcitabine or sunitinib.

In one embodiment the bispecific anti-IGF-1R and anti-ErbB3 antibody has an anti-IGF-1R module selected from the group consisting of SF, P4, M78, and M57. In another embodiment the bispecific anti-IGF-1R and anti-ErbB3 antibody has an anti-ErbB3 module selected from the group consisting of C8, P1, M1.3, M27, P6, and B69. In one embodiment, the bispecific anti-IGF-1R and anti-ErbB3 antibody is P4-G1-M1.3. In another embodiment, the bispecific anti-IGF-1R and anti-ErbB3 antibody is P4-G1-C8.

Also provided are methods of providing treatment of cancer in a human patient comprising co-administering to the patient an effective amount each of a bispecific anti-IGF-1R and anti-ErbB3 antibody and of one or more additional anti-cancer agents, wherein the anti-cancer agent is a PI3K pathway inhibitor, a taxane, an mTOR inhibitor, or an antimetabolite. In some embodiments the anti-cancer agent is an mTOR inhibitor that is selected from the group comprising everolimus, temsirolimus, sirolimus, or ridaforolimus. In other embodiments the anti-cancer agent is the mTOR inhibitor is a pan-mTOR inhibitor chosen from the group consisting of INK128, CC223, OSI207, AZD8055, AZD2014, and Palomid529. In some embodiments the anti-cancer agent is a phosphoinositide-3-kinase (PI3K) inhibitor, e.g., perifosine (KRX-0401), SF1126, CAL101, BKM120, BKM120, XL147, or PX-866. In one embodiment, the PI3K inhibitor is XL147. In another embodiment the anti-cancer agent is an antimetabolite, e.g., gemcitabine, capecitabine, cytarabine, or 5-fluorouracil. In some embodiments, the anti-cancer agent is a taxane, e.g., paclitaxel, nab-paclitaxel, cabazitaxel, or docetaxel. In one embodiment, the one or more anti-cancer agents comprises a taxane and an antimetabolite, e.g., nab-paclitaxel and gemcitabine.

In some embodiments, co-administration of the additional anti-cancer agent or agents has an additive or superadditive effect on suppressing tumor growth, as compared to administration of the bispecific anti-IGF-1R and anti-ErbB3 antibody alone or the one or more additional anti-cancer agents alone, wherein the effect on suppressing tumor growth is measured in a mouse xenograft model using BxPC-3, Caki-1, SK-ES-1, A549, NCI/ADR-RES, BT-474, DU145, or MCF7 cells.

Also provided are compositions for use in the treatment of a cancer, or for the manufacture of a medicament for the treatment of cancer, said composition comprising a bispecific anti-IGF-1R and anti-ErbB3 antibody to be administered to a patient requiring treatment of a cancer, the administration comprising administering to the patient a single loading dose of at least 10 mg/kg of the bispecific antibody followed by administration of one or more maintenance doses given at intervals. The intervals between doses are intervals of at least three days. In some embodiments, the intervals are every fourteen days or every twenty-one days.

In some embodiments, the compositions comprise a loading dose that is greater than the maintenance dose. The loading dose may from 12 mg/kg to 20 mg/kg, from 20 mg/kg to 40 mg/kg, or from 40 mg/kg to 60 mg/kg. In some embodiments the loading dose is about 12 mg/kg, 20 mg/kg, 40 mg/kg, or 60 mg/kg. In other embodiments the maintenance dose is about 6 mg/kg, 12 mg/kg, 20 mg/kg, 30 mg/kg, 40 mg/kg, 50 mg/kg or 60 mg/kg.

In some embodiments the patient has a pancreatic cancer, a KRAS mutant pancreatic cancer, renal cell carcinoma, Ewing's sarcoma, non-small cell lung cancer, gastrointestinal neuroendocrine cancer, estrogen receptor-positive locally advanced or metastatic cancer, ovarian cancer (e.g., high-grade serous ovarian cancer), colorectal cancer, endometrial cancer, or glioblastoma. In one embodiment, the patient has a cancer that is refractory to one or more anti-cancer agents, e.g., gemcitabine or sunitinib.

In one aspect, a patient has a cancer and is selected for treatment with a bispecific anti-IGF-1R and anti-ErbB3 antibody, e.g., MM-141, only if the patient has a serum concentration (level) of free IGF-1 (i.e., IGF-1 in serum that is not bound to an IGF-1 binding protein) that is above the population median level of free IGF-1 for patients with that type of cancer. In one embodiment, the patient has a pancreatic cancer and has a serum level of free IGF-1 that is above the pancreatic cancer population median level of 0.39 ng/ml of free serum IGF-1. Alternately, the serum concentration of free IGF-1 is 1.25, 1.5, 1.75, 2, 2.25, 2.5, 2.75, 3, 3.5, 4, 4.5, 5, 6.5, or 6 times the lower limit of detection for a particular assay, i.e., the assay described in Example 33. Alternately, the patient is treated with MM-141 only if the patient's serum free IGF-1 level meets a cutoff determined for the same type and stage of cancer as the patient. In one embodiment, the cutoff is above the population median level (i.e., the median level in a population of cancer patients with the same type of cancer as the patient). In another embodiment, the cutoff is below the population median level. In one embodiment, the cutoff is about 15%, about 10%, or about 5% below the population median level.

In one embodiment the bispecific anti-IGF-1R and anti-ErbB3 antibody comprises an anti-IGF-1R module selected from the group consisting of SF, P4, M78, and M57. In another embodiment the bispecific anti-IGF-1R and anti-ErbB3 antibody comprises an anti-ErbB3 module selected from the group consisting of C8, P1, M1.3, M27, P6, and B69. In one embodiment, the bispecific anti-IGF-1R and anti-ErbB3 antibody is P4-G1-M1.3. In another embodiment, the bispecific anti-IGF-1R and anti-ErbB3 antibody is P4-G1-C8.

In some embodiments the compositions comprise an effective amount each of a bispecific anti-IGF-1R and anti-ErbB3 antibody and of one or more additional anti-cancer agents, wherein the anti-cancer agent is a PI3K pathway inhibitor, an mTOR inhibitor, or an antimetabolite. In some embodiments the anti-cancer agent is an mTOR inhibitor is selected from the group comprising everolimus, temsirolimus, sirolimus, or ridaforolimus. In other embodiments the mTOR inhibitor is a pan-mTOR inhibitor chosen from the group consisting of INK128, CC223, OSI207, AZD8055, AZD2014, and Palomid529. In some embodiments the anti-cancer agent is a phosphoinositide-3-kinase (PI3K) inhibitor, e.g., perifosine (KRX-0401), SF1126, CAL101, BKM120, BKM120, XL147, or PX-866. In one embodiment, the PI3K inhibitor is XL147. In another embodiment the anti-cancer agent is an antimetabolite, e.g., gemcitabine, capecitabine, cytarabine, or 5-fluorouracil.

In some embodiments the composition comprises a bispecific anti-IGF-1R and anti-ErbB3 antibody and of one or more additional anti-cancer agents, wherein co-administration of the anti-cancer agent or agents has an additive or superadditive effect on suppressing tumor growth, as compared to administration of the bispecific anti-IGF-1R and anti-ErbB3 antibody alone or the one or more additional anti-cancer agents alone, wherein the effect on suppressing tumor growth is measured in a mouse xenograft model using BxPC-3, Caki-1, SK-ES-1, A549, NCI/ADR-RES, BT-474, DU145, or MCF7 cells.

Also provided are kits comprising a therapeutically effective amount of a bispecific anti-IGF-1R and anti-ErbB3 antibody and a pharmaceutically-acceptable carrier. And further comprising instructions to a practitioner, wherein the instructions comprise dosages and administration schedules for the bispecific anti-IGF-1R and anti-ErbB3 antibody. In one embodiment, the kit includes multiple packages each containing a single dose amount of the antibody. In another embodiment, the kit provides infusion devices for administration of the bispecific anti-IGF-1R and anti-ErbB3 antibody. In another embodiment, the kit further comprises an effective amount of at least one additional anti-cancer agent.

### BRIEF DESCRIPTION OF THE DRAWINGS

FIGS. 1A-1D show clinical pharmacodynamic (PD) effects of MM-141 treatment on serum total IGF-1 levels and pharmacokinetic (PK) analysis of serum MM-141 levels for monotherapy dose levels of 6 mg/kg q7d (FIG. 1A), 12 mg/kg q7d (FIG. 1B), 20 mg/kg q7d (FIG. 1C) and 40 mg/kg q14d (FIG. 1D). q7d=qw, q14d=q2w. The y-axis represents either MM-141 in serum in μg/ml (top) or total serum IGF-1 in mg/dL (bottom). The x-axis labeling indicates time in hours in relation to each cycle (C) and week (W) of dosing, with FUP_30D indicating a 30 day follow-up.

FIGS. 2A-2E show the effect of treatment with letrozole, alone and in combination with an anti-ErbB3 antibody, on specified PD markers (per Y-axis labels) in MCF-7Ca breast cancer cell derived xenografts. Quantified western blot data are shown for (FIG. 2A) total ErbB3, (FIG. 2B) phosphorylated FoxO, (FIG. 2C) total IGF-1R, (FIG. 2D) total ErbB2, and (FIG. 2E) phosphorylated IGF-1R.

FIGS. 3A-3C show the effect of treatment with MM-141 and everolimus, alone and in combination, on total IGF-1R and ErbB2 in CAKI-1 xenografts. Quantified western blot data are shown for (FIG. 3A) total IGF-1R and (FIG. 3B) total ErbB2. FIG. 3C shows the activity of MM-141 and everolimus treatment, alone and in combination, in CAKI-1 xenografts.

FIG. 4 shows the effect on the growth of BT-474-M3 xenograft tumors of administration of tamoxifen (Tam) alone, as well as the effects of administration of tamoxifen in a 2-way combination with MM-141 or with everolimus, or in a 3-way combination with MM-141 and everolimus, on the tumors once they exhibited resistance to tamoxifen. The arrow on the X-axis designates the point (day 32) at which combination treatments were initiated, as the tamoxifen-treated tumors had regrown in excess of >20% of their initial tumor volume and so were considered tamoxifen resistant. Tam-treated mice were re-randomized into 4 groups on this day to receive Tam alone, or Tam co-administered with MM-141, with everolimus or with the combination of MM-141 and everolimus.

FIGS. 5A-5C show the effect of treatment with MM-141 and everolimus (each alone, or the two in combination) on phosphorylated AKT and S6 levels in SK-ES-1 Ewing's sarcoma xenografts. Quantified western blot data are shown for blots probed for (FIG. 5A) phosphorylated AKT (Serine 473 specific site), (FIG. 5B) phosphorylated AKT (Threonine 308 specific site), and (FIG. 5C) phosphorylated S6.

FIGS. 6A-6C show the effects of treatment with MM-141 and docetaxel (each alone, or the two in combination), on quantified western blot data for (FIG. 6A) total ErbB3 and (FIG. 6B) total IGF-1R levels, in DU145 prostate cancer-derived xenograft tumors. Also shown (FIG. 6C) are the results of these treatments on the growth of the xenograft tumors.

FIGS. 7A and 7B show the effects of (FIG. 7A) heregulin (HRG) and (FIG. 7B) IGF-1 in reducing the cytotoxic effect of paclitaxel in BxPC-3 pancreatic cancer cells in vitro, and the effect of added MM-141 in restoring paclitaxel sensitivity in the presence of these ligands.

FIGS. 8A and 8B show the effects of a combination of HRG and IGF-1 ligands in reducing the cytotoxic effect of gemcitabine and paclitaxel in (FIG. 8A) BxPC-3 KRAS wild type and (FIG. 8B) CFPAC-1 KRAS mutant pancreatic cancer cells in vitro, and the effect of added MM-141 in restoring gemcitabine and paclitaxel sensitivity in the presence of these ligands. Y axis “CTG luminescence signal” indicates raw luminescence values representative of cell viability per CTG assay.

FIGS. 9A-9C shows the PD effects of treatment with MM-141 and gemcitabine, alone and in combination, on (FIG. 9A) total IGF-1R and (FIG. 9B) total ErbB3 levels in BxPC-3 derived xenograft tumors. Also shown (FIG. 9C) are the results of the treatments on the growth of the xenograft tumors, and the effects of adding MM-141 to the gemcitabine regimen after the development of gemcitabine resistance.

FIG. 10 shows the effects of treatment with nab-paclitaxel and gemcitabine in a 2-way combination and in a 3-way combination with MM-141 on the growth of HPAF-II pancreatic xenograft tumors. Nab-paclitaxel and gemcitabine are co-administered at two different doses, alone and in combination with MM-141.

FIG. 11 shows the effects of treatment with nab-paclitaxel and gemcitabine in a triple combination regimen with MM-141 on the growth of patient-derived pancreatic primary xenograft tumors.

FIGS. 12A and 12B show the effects of MM-141 and sorafenib, either alone or in combination, on HepG2 hepatocellular carcinoma cells in vitro. Quantitative western blot data are shown for (FIG. 12A) total ErbB3 and (FIG. 12B) phosphorylated AKT.

FIG. 13 shows the effects of treatment with docetaxel, alone and in combination with MM-141, on the viability of BxPC-3 pancreatic carcinoma cells in vitro, measured using a CTG assay.

FIGS. 14A and 14B show the in vitro PD effects of treatment with MM-141 and trametinib (GSK-1120212), alone and in combination, on pAKT levels in (FIG. 14A) BxPC-3 and (FIG. 14B) KP4 pancreatic cancer cell lines.

FIGS. 15A-15C show the effect of MM-141 and its component IGF-1R antibody on proliferation of Bx-PC3 pancreatic cancer cells grown either in low serum alone or with exogenous IGF-1 or heregulin (HRG) added. Y axis “% cell growth” indicates viability per CTG assay.

FIGS. 16A-16E show the inhibitory effects of MM-141 on anchorage-dependent (FIG. 2D, FIG. 16A) and anchorage-independent (FIG. 3D, FIG. 16B) proliferation of ovarian cancer cell lines, as measured by CTG assay. FIGS. 16C-16E show that MM-141 blocks IGF-1 and HRG growth-factor-induced proliferation in ovarian cancer cell lines in 3D in vitro assays in PEA1 cells (FIG. 16C), PEA2 cells (FIG. 16D), and OvCAR8 cells (FIG. 16E).

FIGS. 17A and 17B show the effects of IGF-1 or HRG in reducing the cytotoxic effect of paclitaxel in platinum-sensitive (s) and platinum-resistant (r) ovarian cancer cells in vitro (Peol (s), Peo4 (r), OvCAR8 (s) in FIG. 17A; PEA1 (s), PEA2 (s), and Ov90 in FIG. 17B) and the effect of added MM-141 in restoring paclitaxel sensitivity in the presence of these ligands.

FIG. 18A-18F show the effect of MM-141 on basal and growth factor (IGF-1, FIGS. 18A-C, and HRG, FIGS. 18D-18F)-induced levels and activation states of IGF-1R, ErbB3, AKT and ERK cells in a selection of ovarian cancer cell lines in vitro. FIGS. 18A-F show PEA1, PEA2, OvCAR5, Peol, Peo4, and PEA2 cells, respectively.

FIG. 19 shows the effect of ligand stimulation on AKT activation in a panel of pancreatic cancer cell lines. Phosphorylated AKT (pAKT) levels per cell line, as measured by ELISA, are represented as a heatmap.

FIG. 20 shows the effect of MM-141 treatment on HRG and IGF-1-induced phosphorylation of AKT in a panel of pancreatic cancer cell lines. Bar graphs represent pAKT levels post-treatment with HRG and IGF-1, with or without MM-141.

FIGS. 21A and 21B show the effects of treatment with MM-141 on both (FIG. 21A) ErbB3 and (FIG. 21B) IGF-1R levels in CFPAC-1 pancreatic cells. Bar graphs represent levels of ErbB3 and IGF-1R post-treatment with MM-141, or mono-specific antibodies targeting ErbB3 or IGF-1R.

FIGS. 22A and 22B show the effects of treatment with MM-141 in combination with (FIG. 22A) gemcitabine or (FIG. 22B) paclitaxel in the presence of HRG and IGF-1 ligands on CFPAC-1 pancreatic cell proliferation. Bar graphs represent proliferation of CFPAC-1 cells, normalized to untreated control at 1.

FIGS. 23A and 23B show the in vitro PD effects of treatment with gemcitabine, paclitaxel or SN-38 on (FIG. 23A) ErbB3 and (FIG. 23B) IGF-1R levels in CFPAC-1 pancreatic cells.

FIGS. 24A and 24B show the effects of treatment with gemcitabine on pAKT (Ser473) levels, in the presence or absence of MM-141 on (FIG. 24A) HRG- or (FIG. 24B) IGF-1 stimulated CFPAC-1 cells.

FIGS. 25A and 25B show the effects of treatment with paclitaxel on pAKT (Ser473) levels, in the presence or absence of MM-141 on (FIG. 25A) HRG- or (FIG. 25B) IGF-1 stimulated CFPAC-1 cells.

FIGS. 26A and 26B show the effects of treatment with gemcitabine and nab-paclitaxel alone and in combination with MM-141, on long-term growth of (FIG. 26A) HPAF-II KRAS mutant, and (FIG. 26B) CFPAC-1 KRAS mutant pancreatic xenografts.

FIG. 27 shows the effects of treatment with MM-141 and nab-paclitaxel, alone and in combination, on long-term growth on CFPAC-1 KRAS mutant pancreatic xenografts.

FIGS. 28A and 28B shows the effect of treatment with nab-paclitaxel (Abx) and gemcitabine (gem), alone or in combination with MM-141, on membrane receptor levels in HPAF-II xenograft tumors. Quantified immunoblot data are shown for (FIG. 28A) total IGF-1R and (FIG. 28B) total ErbB3.

FIGS. 29A and 29B show the effect of nab-paclitaxel (Abx) and gemcitabine (gem) treatment, alone or in combination with MM-141, on intracellular signaling effector levels in HPAF-II xenografts. Quantified immunoblot data are shown for (FIG. 29A) phospho-4ebp-1 (S65) and (FIG. 29B) phospho-S6 (S240/244).

FIGS. 30A and 30B show the effect of treatment with nab-paclitaxel (Nab) and gemcitabine (gem), alone or in combination with MM-141, on membrane receptor levels in HPAF-II xenograft tumors. Quantified immunoblot data are shown for (FIG. 30A) total IGF-1R and (FIG. 30B) total ErbB3.

FIGS. 31A and 31B show the effect of treatment with nab-paclitaxel (Nab) and gemcitabine (Gem), alone or in combination with MM-141, on membrane receptor levels in HPAF-II xenograft tumors. Quantified immunoblot data are shown for (FIG. 31A) total IGF-1R and (FIG. 31B) total ErbB3.

FIG. 32 shows the pre- (top panels) and post- (bottom panels) MM-141 treatment levels of ErbB3 (left panels) and IGF-1R (right panels), as detected by immunohistochemistry, in hepatocellular carcinoma tumor biopsies taken from a patient enrolled in an MM-141 Phase 1 clinical trial.

FIGS. 33A-33D show the effects of MM-141 on surface expression levels of IGF-1R (FIG. 33A) and ErbB3 (FIG. 33B) compared to the effects of a monospecific IGF-1R antibody and a monospecific ErbB3 antibody, as measured by ELISA. In addition, treatment with MM-141 leads to increased degradation of IGF-1R (FIG. 33C) and ErbB3 (FIG. 33D) receptors, as evidenced by enhanced receptor ubiquitination, measured using immunoprecipitation and immunoblotting assays in vitro.

FIG. 34 shows the effects of gemcitabine and paclitaxel treatment on HRG mRNA expression in CFPAC-1 pancreatic cancer cells in vitro.

FIGS. 35A and 35B show the distribution of free IGF-1 in serum (i.e., IGF-1 not bound by one or more of six IGF-1 binding proteins). FIG. 35A shows the distribution in serum taken from Stage 3 and Stage 4 pancreatic cancer patients. Each column represents a single serum sample. FIG. 35B shows that Phase 1 breast cancer patients who have a level of free serum IGF-1 above a cutpoint are able to stay on study longer, and thus receive more therapeutic doses of MM-141, than patients whose level of free serum IGF-1 was below the cutpoint.

FIG. 36 shows modeling of the steady state exposure of MM-141 administered at different dosing schedules. Average, maximal and minimal steady state concentrations of MM-141 were modeled on the basis of Phase 1 PK data. The 2.8 g Q2W regimen was indicated to have similar exposures to the 40 mg/kg Q2W regimen and the 2.24 g Q2W regimen was indicated to yield smaller exposures than the 20 mg/kg QW regimen.

### DETAILED DESCRIPTION

### Methods and Compositions

Methods of combination therapy and combination compositions for treating cancer in a patient are provided. In these methods, the cancer patient is treated with both a bispecific anti-IGF-1R and anti-ErbB3 antibody and one or more additional anti-cancer agents selected, e.g., from an mTOR inhibitor, a PI3K inhibitor, and an antimetabolite.

The terms “combination therapy,” “co-administration,” “co-administered” or “concurrent administration” (or minor variations of these terms) include simultaneous administration of at least two therapeutic agents to a patient or their sequential administration within a time period during which the first administered therapeutic agent is still present in the patient (e.g., in the patient's plasma or serum) when the second administered therapeutic agent is administered.

The term “monotherapy” refers to administering a single drug to treat a disease or disorder in the absence of co-administration of any other therapeutic agent that is being administered to treat the same disease or disorder.

“Additional anti-cancer agent” is used herein to indicate any drug that is useful for the treatment of a malignant pancreatic tumor other than a drug that inhibits heregulin binding to ErbB2/ErbB3 heterodimer.

“Dosage” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., as one or more administrations, e.g., either or both of orally (e.g., as one, two, three or more pills, capsules, etc.) or injected (e.g., as a bolus or infusion). Dosage sizes may also relate to doses that are administered continuously (e.g., as an intravenous infusion over a period of minutes or hours). Such parameters further include frequency of administration of separate doses, which frequency may change over time.

“Dose” refers to an amount of a drug given in a single administration.

“Effective amount” refers to an amount (administered in one or more doses) of an antibody, protein or additional therapeutic agent, which amount is sufficient to provide effective treatment.

“ErbB3” and “HER3” refer to ErbB3 protein, as described in U.S. Pat. No. 5,480,968. The human ErbB3 protein sequence is shown in SEQ ID NO:4 of U.S. Pat. No. 5,480,968, wherein the first 19 amino acids (aas) correspond to the leader sequence that is cleaved from the mature protein. ErbB3 is a member of the ErbB family of receptors, other members of which include ErbB1 (EGFR), ErbB2 (HER2/Neu) and ErbB4. While ErbB3 itself lacks tyrosine kinase activity, but is itself phosphorylated upon dimerization of ErbB3 with another ErbB family receptor, e.g., ErbB1 (EGFR), ErbB2 and ErbB4, which are receptor tyrosine kinases. Ligands for the ErbB family receptors include heregulin (HRG), betacellulin (BTC), epidermal growth factor (EGF), heparin-binding epidermal growth factor (HB-EGF), transforming growth factor alpha (TGF-α), amphiregulin (AR), epigen (EPG) and epiregulin (EPR). The aa sequence of human ErbB3 is provided at Genbank Accession No. NP_001973.2 (receptor tyrosine-protein kinase erbB-3 isoform 1 precursor) and is assigned Gene ID: 2065.

“IGF-1R” or “IGF1R” refers to the receptor for insulin-like growth factor 1 (IGF-1, formerly known as somatomedin C). IGF-1R also binds to, and is activated by, insulin-like growth factor 2 (IGF-2). IGF1-R is a receptor tyrosine kinase, which, upon activation by IGF-1 or IGF-2, is auto-phosphorylated. The aa sequence of human IGF-1R precursor is provided at Genbank Accession No. NP_000866 and is assigned Gene ID: 3480.

“Module” refers to a structurally and/or functionally distinct part of a PBA, such a binding site (e.g., an scFv domain or a Fab domain) and the Ig constant domain. Modules provided herein can be rearranged (by recombining sequences encoding them, either by recombining nucleic acids or by complete or fractional de novo synthesis of new polynucleotides) in numerous combinations with other modules to produce a wide variety of PBAs as disclosed herein. For example, an “SF” module refers to the binding site “SF,” i.e., comprising at least the CDRs of the SF VH and SF VL domains. A “C8” module refers to the binding site “C8.”

“PBA” refers to a polyvalent bispecific antibody, an artificial hybrid protein comprising at least two different binding moieties or domains and thus at least two different binding sites (e.g., two different antibody binding sites), wherein one or more of the pluralities of the binding sites are covalently linked, e.g., via peptide bonds, to each other. A preferred PBA described herein is an anti-IGF-1R+anti-ErbB3 PBA (e.g., as disclosed in U.S. Pat. No. 8,476,409), which is a polyvalent bispecific antibody that comprises one or more first binding sites binding specifically to human IGF-1R protein, and one or more second binding sites binding specifically to human ErbB3 protein. An anti-IGF-1R+anti-ErbB3 PBA is so named regardless of the relative orientations of the anti-IGF-1R and anti-ErbB3 binding sites in the molecule, whereas when the PBA name comprises two antigens separated by a slash (/) the antigen to the left of the slash is amino terminal to the antigen to the right of the slash. A PBA may be a bivalent binding protein, a trivalent binding protein, a tetravalent binding protein or a binding protein with more than 4 binding sites. An exemplary PBA is a tetravalent bispecific antibody, i.e., an antibody that has 4 binding sites, but binds to only two different antigens or epitopes. Exemplary bispecific antibodies are tetravalent “anti-IGF-1R/anti-ErbB3” PBAs and “anti-ErbB3/anti-IGF-1R” PBAs. Typically the N-terminal binding sites of a tetravalent PBA are Fabs and the C-terminal binding sites are scFvs. Exemplary IGF-1R+ErbB3 PBAs comprising IgG1 constant regions each comprise two joined essentially identical subunits, each subunit comprising a heavy and a light chain that are disulfide bonded to each other, (SEQ ID NOs hereinafter refer to sequences set forth in U.S. Pat. No. 8,476,409, which is herein incorporated by reference in its entirety) e.g., M7-G1-M78 (SEQ ID NO: 284 and SEQ ID NO: 262 are the heavy and light chain, respectively), P4-G1-M1.3 (SEQ ID NO: 226 and SEQ ID NO: 204 are the heavy and light chain, respectively), and P4-G1-C8 (SEQ ID NO: 222 and SEQ ID NO: 204 are the heavy and light chain, respectively), are exemplary embodiments of such IgG1-(scFv)₂ proteins. When the immunoglobulin constant regions are those of IgG2, the protein is referred to as an IgG2-(scFv)₂. Other exemplary IGF-1R+ErbB3 PBAs comprising IgG1 constant regions include (as described in U.S. Pat. No. 8,476,409) SF-G1-P1, SF-G1-M1.3, SF-G1-M27, SF-G1-P6, SF-G1-B69, P4-G1-C8, P4-G1-P1, P4-G1-M1.3, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, P1-G1-M76.

The heavy and light chain sequences of M7-G1-M78, P4-G1-M1.3, and P4-G1-C8 are listed below:

M7-G1-M78 Heavy chain
(SEQ ID NO: 1)
EVQLVESGGGLVQPGRSLRLSCAASGFTFDDYAMHWVRQAPGKGLEWVSG
ISWDSGSVGYADSVKGRFTISRDNAKNSLYLQMNSLRAEDTALYYCARDL
GYNQWwEGFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GGGGGSGGGGSGGGGSEVQLLQSGGGLVQPGGSLRLSCAASGFDFSSYPM
HWVRQAPGKGLEWVGSISSSGGATPYADSVKGRFTISRDNSKNTLYLQMN
SLRPEDTAVYYCAKDFYTILTGNAFDMWGQGTSVTVSSASTGGGGSGGGG
SGGGGSGGGGSDIQMTQSPSSLSASLGDRVTITCRASQGISSYLAWYQQK
PGKAPKLLIYASSTRQSGVPSRFSGSGSGTDFTLTISSLQPEDSGTYYCQ
QYWAFPLTFGGGTKVEIKRT
M7-G1-M78 Light chain
(SEQ ID NO: 2)
SSELTQDPAVSVALGQTVRITCQGDSLRSYYASWYQQKPGQAPVLVIYGK
NNRPSGIPDRFSGSSSGNTASLTITGAQAEDEADYYCNSRDTPGNKWVFG
GGTKVTVIGQPKAAPSVTLFPPSSEELQANKATLVCLVSDFYPGAVTVAW
KADGSPVKVGVETTKPSKQSNNKYAASSYLSLTPEQWKSHRSYSCRVTHE
GSTVEKTVAPAECS
P4-G1-M1.3 Heavy chain
(SEQ ID NO: 3)
EVQLLQSGGGLVQPGGSLRLSCAASGFMFSRYPMHWVRQAPGKGLEWVGS
ISGSGGATPYADSVKGRFTISRDNSKNTLYLQMNSLRAEDTAVYYCAKDF
YQILTGNAFDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GGGGGSGGGGSGGGGSQVQLVQSGGGLVQPGGSLRLSCAASGFTFDDYAM
HWVRQAPGKGLEWVAGISWDSGSTGYADSVKGRFTISRDNAKNSLYLQMN
SLRAEDTALYYCARDLGAYQWVEGFDYWGQGTLVTVSSASTGGGGSGGGG
SGGGGSGGGGSSYELTQDPAVSVALGQTVRITCQGDSLRSYYASWYQQKP
GQAPVLVIYGKNNRPSGIPDRFSGSTSGNSASLTITGAQAEDEADYYCNS
RDSPGNQWVFGGGTKVTVLG
P4-G1-M1.3 and P4-G1-C8 Light chain
(SEQ ID NO: 4)
DIQMTQSPSSLSASLGDRVTITCRASQGISSYLAWYQQKPGKAPKLLIYA
KSTLQSGVPSRFSGSGSGTDFTLTISSLQPEDSATYYCQQYWTFPLTFGG
GTKVEIKRTVAAPSVFIFPPSDEQLKSGTASVVCLLNNFYPREAKVQWKV
DNALQSGNSQESVTEQDSKDStYSLSSTLTLSKADYEKHKVYACEVTHQG
LSSPVTKSFNRGEC
P4-G1-C8 Heavy chain
(SEQ ID NO: 5)
EVQLLQSGGGLVQPGGSLRLSCAASGFMFSRYPMHWVRQAPGKGLEWVGS
ISGSGGATPYADSVKGRFTISRDNSKNTLYLQMNSLRAEDTAVYYCAKDF
YQILTGNAFDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GGGGGSGGGGSGGGGSQVQLVQSGGGLVQPGGSLRLSCAASGFTFDDYAM
HWVRQAPGKGLEWVAGISWNSGSIGYADSVKGRFTISRDNAKNSLYLQMN
SLRPEDTAVYYCARDLGYNQWVEGFDYWGQGTLVTVSSASTGGGGSGGGG
SGGGGSGGGGSSYELTQDPAVSVALGQTVRITCQGDSLRSYYASWYQQKP
GQAPVLVIYGKNNRPSGIPDRFSGSTSGNSASLTITGAQAEDEADYYCNS
RDSSGNHWVFGGGTKVTVLG

The term “MM-141” refers to polyvalent bispecific antibody P4-G1-M1.3 having two pairs of polypeptide chains, each pair of said two pairs comprising a heavy chain joined to a light chain by at least one heavy-light chain bond, wherein each light chain comprises the amino acid sequence set forth in SEQ ID NO:204 and each heavy chain comprises the amino acid sequence set forth in SEQ ID NO:226, wherein SEQ ID NOs: 204 and 226 are those as set forth in U.S. Pat. No. 8,476,409 (which is herein incorporated by reference in its entirety) and above.

Combination Therapies with Additional Anti-Cancer Agents

As herein provided, PBAs (e.g., P4-G1-M1.3) are co-administered with one or more additional anti-cancer agents (e.g., an mTOR inhibitor, a PI3K inhibitor, or an antimetabolite), to provide effective treatment to human patients having a cancer (e.g., pancreatic, ovarian, lung, colon, head and neck, and esophageal cancers).

Additional anti-cancer agents suitable for combination with anti-ErbB3 antibodies may include, but are not limited to, pyrimidine antimetabolites, mTOR inhibitors, pan-mTOR inhibitors, phosphoinositide-3-kinase (PI3K) inhibitors, MEK inhibitors, taxanes, and nanoliposomal irinotecan (e.g., MM-398).

Gemcitabine (Gemzar®) is indicated as a first line therapy for pancreatic adenocarcinoma and is also used in various combinations to treat ovarian, breast and non-small-cell lung cancers. Gemcitabine HCl is 2′-deoxy-2′,2′-difluorocytidine monohydrochloride (-isomer) (MW=299.66) and is administered parenterally, typically by i.v. infusion.

Cytarabine (Cytosar-U® or Depocyt®) is mainly used in the treatment of acute myeloid leukemia, acute lymphocytic leukemia and in lymphomas. Cytarabine is rapidly deaminated in the body into the inactive uracil derivative and therefore is often given by continuous intravenous infusion.

Temsirolimus (Torisel®) is an mTOR inhibitor that is administered parenterally, typically by i.v. infusion and is used to treat advanced renal cell carcinoma.

Everolimus (Afinitor®), a 40-O-(2-hydroxyethyl) derivative of sirolimus, is an mTOR inhibitor that is administered orally and is used to treat progressive neuroendocrine tumors of pancreatic origin (PNET) in patients with unresectable, locally advanced or metastatic disease.

Sirolimus (rapamycin, Rapamune®) is an mTOR inhibitor that has been shown to inhibit the progression of dermal Kaposi's sarcoma in patients with renal transplants.

Ridaforolimus (also known as AP23573 and MK-8669) is an investigational mTOR inhibitor being tested for treatment of metastatic soft tissue, breast cancer and bone sarcomas (CAS No. 572924-54-0).

INK128 is of a class of mTOR inhibitors that competes with ATP binding site on mTOR, and inhibits activity of TOR complexes 1 and 2 (TORC1/TORC2). It is currently being investigated in a number of clinical trials for solid tumors (CAS No. 1224844-38-5).

CC-223 (TORKi®) is an investigational, orally available inhibitor of mTOR that inhibits activity of TOR complexes 1 and 2 (TORC1/TORC2). It is currently being investigated in clinical trials.

OSI-027 is a selective and potent dual inhibitor of mTORC1 and mTORC2 with more than 100-fold selectivity observed for mTOR than PI3Kα, PI3Kβ, PI3Kγ or DNA-PK. It is currently in clinical trials, e.g., for solid tumors or lymphomas (CAS No. 936890-98-1).

AZD8055 is an ATP-competitive mTOR inhibitor with excellent selectivity (˜1,000-fold) against PI3K isoforms and ATM/DNA-PK. It is currently in clinical trials, e.g., for hepatocellular carcinoma, malignant glioma (CAS No. 1009298-09-2).

AZD2014 inhibits both the TORC1 and TORC2 complexes, and is currently undergoing clinical trials for a variety of cancers (CAS No. 1009298-59-2).

Palomid 529 (P529) inhibits both the TORC1 and TORC2 complexes, and reduces phosphorylation of pAktS473, pGSK3βS9, and pS6. It is currently being investigated in clinical trials (CAS No. 914913-88-5).

5-Fluorouracil (5-FU Adrucil®, Carac®, Efudix®, Efudex® and Fluoroplex®) is a pyrimidine analog that works through irreversible inhibition of thymidylate synthase. 5-Fluorouracil has been given systemically for anal, breast, colorectal, oesophageal, stomach, pancreatic and skin cancers (especially head and neck cancers).

Capecitabine (Xeloda®) is an orally administered systemic prodrug of 5′-deoxy-5-fluorouridine (5′-DFUR) which is converted to 5-fluorouracil.

Docetaxel (Taxotere®) is an anti-mitotic chemotherapy used for the treatment of breast, advanced non-small cell lung, metastatic androgen-independent prostate, advanced gastric and locally advanced head and neck cancers.

Paclitaxel (Taxol®) is an anti-mitotic chemotherapy used for the treatment of lung, ovarian, breast and head and neck cancers.

Perifosine (previously KRX-0401) is an AKT inhibitor that targets the plekstrin homology domain of Akt. It is currently being investigated in a number of clinical trials (CAS No. 157716-52-4)

SF1126 selectively inhibits all isoforms of phosphoinositide-3-kinase (PI3K) and other members of the PI3K superfamily, such as the mammalian target of rapamycin (mTOR) and DNA-PK. It is currently being investigated in a number of clinical trials (CAS 936487-67-1).

CAL101 (Idelalisib, GS-1101) is a PI3K inhibitor is currently being investigated in a number of clinical trials for leukemias and lymphomas (CAS No. 870281-82-6).

BKM120 (Buparlisib) is a PI3K inhibitor currently being investigated in clinical trials, e.g., for nn-small cell lung cancer (CAS No. 944396-07-0).

XL147 is a selective and reversible class I PI3K inhibitor currently being investigated in clinical trials, e.g., for malignant neoplasms (CAS No. 956958-53-5).

PX-866 (sonolisib) is a small-molecule wortmannin analog inhibitor of the alpha, gamma, and delta isoforms of phosphoinositide 3-kinase (PI3K) with potential antineoplastic activity, and is currently being investigated in clinical trials (CAS No. 502632-66-8).

Sorafenib (Nexavar®) is a small molecule inhibitor of multiple tyrosine kinases (including VEGFR and PDGFR) and Raf kinases (an exemplary “multikinase inhibitor”) used for treatment of advanced renal cell carcinoma (RCC) and advanced primary liver cancer (hepatocellular carcinoma, HCC) (CAS No. 284461-73-0).

Trametinib (GSK-1120212) is a small molecule inhibitor of the MEK protein currently in clinical trials for the treatment of several cancers including pancreatic, melanoma, breast and non-small cell lung (CAS No. 871700-17-3).

Selumetinib (AZD6244) is a potent, highly selective MEK1 inhibitor, currently in clinical trials for various types of cancer, including non-small cell lung cancer (CAS No. 606143-52-6).

Refametinib (RDEA119, BAY86-9766) is a potent, ATP non-competitive and highly selective inhibitor of MEK1 and MEK2. It is currently being investigated in clinical trials for the treatment of various cancers, including hepatocellular carcinoma (CAS No. 923032-37-5; formal name: N-[3,4-difluoro-2-[(2-fluoro-4-iodophenyl)amino]-6-methoxyphenyl]-1-[(2S)-2,3-dihydroxypropyl]-cyclopropanesulfonamide).

Vemurafenib (Zelboraf®) is a small molecule inhibitor of B-Raf in patients whose cancer cells harbor a V600E B-Raf mutation. Vemurafenib is currently approved for treatment of late-stage, unresectable, and metastatic melanoma (CAS No. 918504-65-1).

Nab-paclitaxel (Abraxane®) is a nanoparticulate albumin-bound formulation of paclitaxel (Paclitaxel CAS No. 33069-62-4).

Nanoliposomal irinotecan (irinotecan sucrosofate liposome injection: MM-398) is a stable nanoliposomal formulation of irinotecan. MM-398 is described, e.g., in U.S. Pat. No. 8,147,867. MM-398 may be administered, for example, on day 1 of the cycle at a dose of 120 mg/m2, except if the patient is homozygous for allele UGT1A1*, wherein nanoliposomal irinotecan is administered on day 1 of cycle 1 at a dose of 80 mg/m². The required amount of MM-398 may be diluted, e.g., in 500 mL of 5% dextrose injection USP and infused over a 90 minute period.

### Outcomes

As shown in the Examples herein, co-administration of an anti-ErbB3 antibody with one or more additional therapeutic agents (e.g., everolimus, temsirolimus, sirolimus, XL147, gemcitabine, 5-fluorouracil, and cytarabine) provides improved efficacy compared to treatment with the antibody alone or with the one or more additional therapeutic agents in the absence of antibody therapy. Preferably, a combination of an anti-ErbB3 antibody with one or more additional therapeutic agents exhibits therapeutic synergy.

“Therapeutic synergy” refers to a phenomenon where treatment of patients with a combination of therapeutic agents manifests a therapeutically superior outcome to the outcome achieved by each individual constituent of the combination used at its optimum dose (T. H. Corbett et al., 1982, Cancer Treatment Reports, 66, 1187). In this context a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone.

Thus, in combination, the components of such combinations have an additive or superadditive effect on suppressing tumor growth, as compared to monotherapy with the PBA or treatment with the chemotherapeutic(s) in the absence of antibody therapy. By “additive” is meant a result that is greater in extent (e.g., in the degree of reduction of tumor mitotic index or of tumor growth or in the degree of tumor shrinkage or the frequency and/or duration of symptom-free or symptom-reduced periods) than the best separate result achieved by monotherapy with each individual component, while “superadditive” is used to indicate a result that exceeds in extent the sum of such separate results. In one embodiment, the additive effect is measured as slowing or stopping of tumor growth. The additive effect can also be measured as, e.g., reduction in size of a tumor, reduction of tumor mitotic index, reduction in number of metastatic lesions over time, increase in overall response rate, or increase in median or overall survival.

One non-limiting example of a measure by which effectiveness of a therapeutic treatment can be quantified is by calculating the log 10 cell kill, which is determined according to the following equation:

log 10 cell kill=TC(days)/3.32×Td

in which T C represents the delay in growth of the cells, which is the average time, in days, for the tumors of the treated group (T) and the tumors of the control group (C) to have reached a predetermined value (1 g, or 10 mL, for example), and Td represents the time, in days necessary for the volume of the tumor to double in the control animals. When applying this measure, a product is considered to be active if log 10 cell kill is greater than or equal to 0.7 and a product is considered to be very active if log 10 cell kill is greater than 2.8. Using this measure, a combination, used at its own maximum tolerated dose, in which each of the constituents is present at a dose generally less than or equal to its maximum tolerated dose, exhibits therapeutic synergy when the log 10 cell kill is greater than the value of the log 10 cell kill of the best constituent when it is administered alone. In an exemplary case, the log 10 cell kill of the combination exceeds the value of the log 10 cell kill of the best constituent of the combination by at least 0.1 log cell kill, at least 0.5 log cell kill, or at least 1.0 log cell kill.

### Kits and Unit Dosage Forms

Further provided are kits that include a pharmaceutical composition containing a bispecific anti-IGF-1R and anti-ErbB3 antibody, including a pharmaceutically-acceptable carrier, in a therapeutically effective amount adapted for use in the preceding methods. The kits include instructions to allow a practitioner (e.g., a physician, nurse, or physician's assistant) to administer the composition contained therein to treat an ErbB2 expressing cancer.

Preferably, the kits include multiple packages of the single-dose pharmaceutical composition(s) containing an effective amount of a bispecific anti-IGF-1R and anti-ErbB3 antibody for a single administration in accordance with the methods provided above. Optionally, instruments or devices necessary for administering the pharmaceutical composition(s) may be included in the kits. For instance, a kit may provide one or more pre-filled syringes containing an amount of a bispecific anti-IGF-1R and anti-ErbB3 antibody that is about 100 times the dose in mg/kg indicated for administration in the above methods.

Furthermore, the kits may also include additional components such as instructions or administration schedules for a patient suffering from a cancer to use the pharmaceutical composition(s) containing a bispecific anti-IGF-1R and anti-ErbB3 antibody.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### EXAMPLES

The following Examples should not be construed as limiting the scope of this disclosure. Unless specifically stated, all commercial antibodies used for western blotting in the following Examples were provided by Cell Signaling Technologies and, in all western blots, signal was normalized to β-Actin levels detected by western blot as a loading control. Where treatments of cancer in patients are set forth in the Examples below, the cancer to be treated is pancreatic cancer, ovarian cancer (e.g., high-grade serous ovarian cancer), sorafenib-naive or sorafenib-refractory hepatocellular carcinoma, parathyroid cancer, sarcoma, lung cancer or breast cancer. Where measured in xenograft studies, tumors were measured bi-weekly using digital calipers, and volumes (mm³) were calculated according to the formula: π/6×(length×width×width).

### Example 1

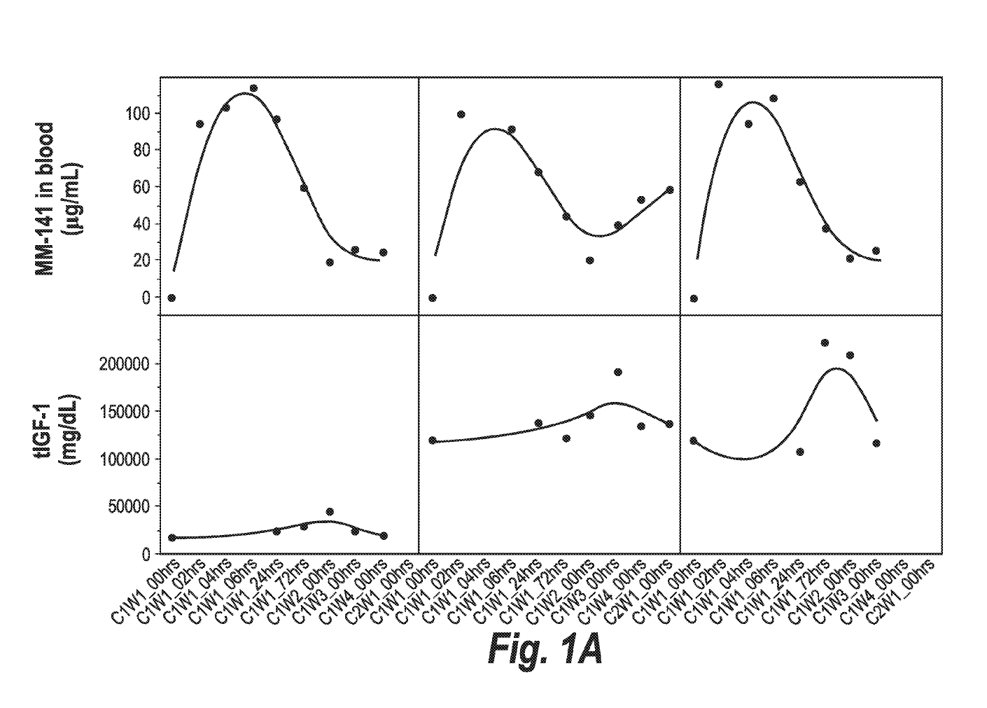

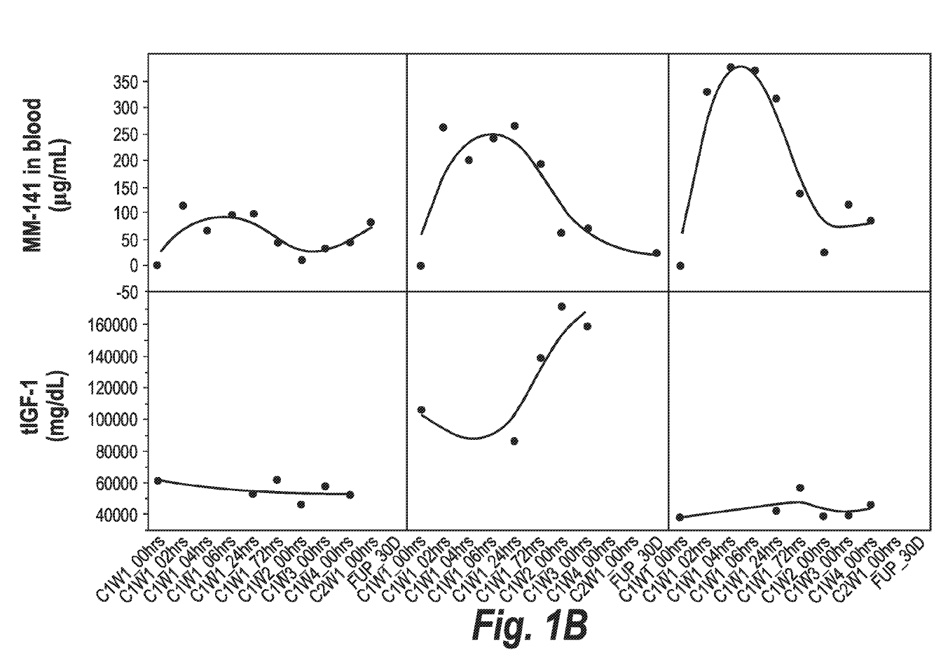

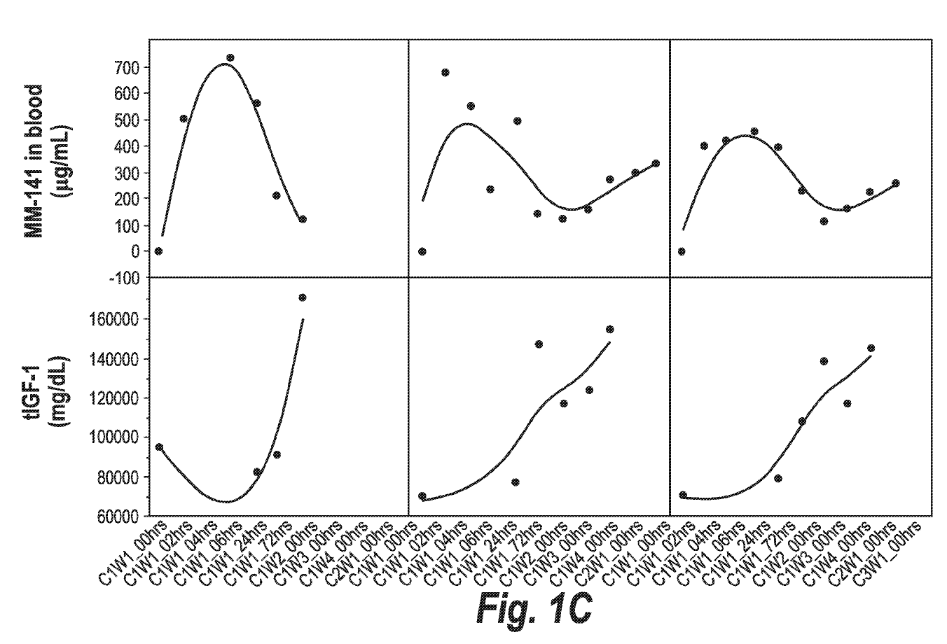

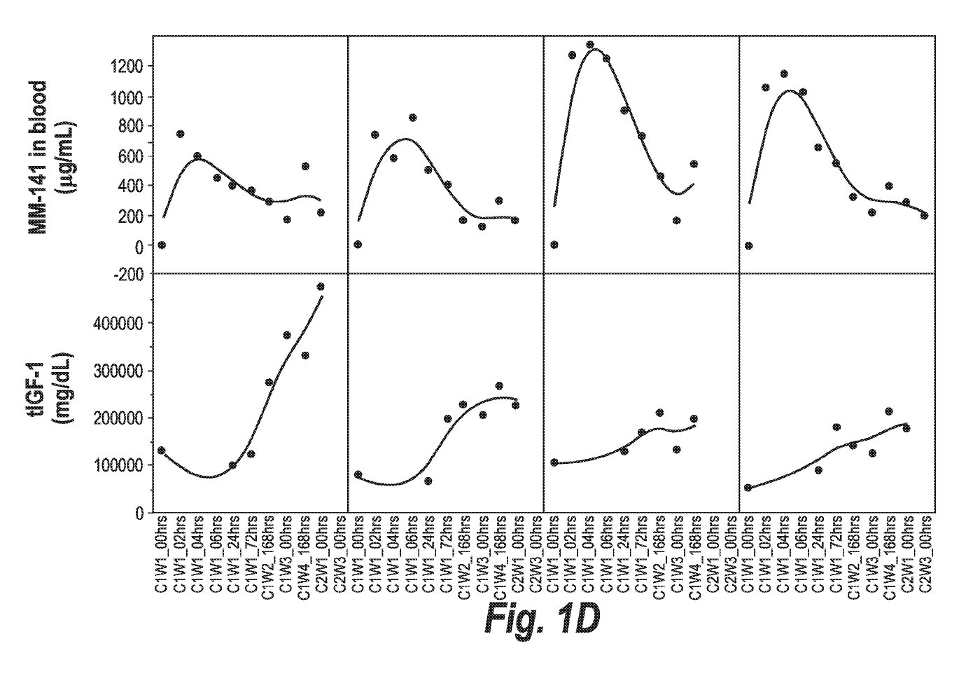

This Example discloses the results of treatment of patients with solid tumors in a Phase 1 dose escalation study with MM-141 administered as monotherapy. 15 patients were dosed with MM-141 monotherapy at 6 mg/kg (n=3), 12 mg/kg (n=4), 20 mg/kg (n=4) q7d, or 40 mg/kg (n=4) q14d. No dose-limiting toxicities were observed at any of these dose levels. Adverse events that were reported with a frequency>15% included: vomiting (7/15), nausea (6/15), fatigue (4/15), abdominal pain (4/15), increased AP (4/15), dyspnea (4/15), diarrhea (3/15), anemia (3/15), increased AST (3/15), and rash (3/15). Pharmacokinetic (PK), and pharmacodynamic (PD) analysis of MM-141 as monotherapy are shown in FIGS. 1(A-D). Half-lives (T for each dose level were in the ranges of 2.4-6.3 days (6 mg/kg), 2.1-2.9 days (12 mg/kg), 3.3-3.4 days (20 mg/kg) and 3.2-9.9 days (40 mg/kg). Increases in serum total IGF-1 levels in response to MM-141 dosing were seen in each cohort with greater magnitude as dosing escalated. Total IGF-1 increased approximately two-fold in 1/3 patient samples analyzed in each of the 6 mg/kg and 12 mg/kg cohorts. At 20 mg/kg all patient samples exhibited approximately a two-fold increase in total IGF-1, and at 40 mg/kg all patient samples exhibited approximately two to four fold increases in total IGF-1. The safety, tolerability, PK and PD profiles support weekly and biweekly MM-141 dosing. Disease stabilization was observed in patients with Ewing's Sarcoma (1) and parotid gland carcinoma (1). Recommended dose levels for MM-141 Phase 2 study were established as 20 mg/kg q7d and 40 mg/kg q14d.

Serum for PK and PD analysis was prepared by drawing whole blood into red top tubes, clotting 30 minutes at 4-8° C. and spinning down in a refrigerated centrifuge. Serum was aliquotted and frozen immediately after centrifugation. PD analysis of total IGF-1 in serum was performed using Human IGF-I Quantikine® ELISA Kit (R&D Systems, Minneapolis, Minn.) according to the manufacturer's instructions. For the PK analysis, in brief, ELISA plates were plates were coated with IGF-1R (R&D Systems) in PBS and incubated overnight at 4° C. Plates were washed, blocked, and then samples and standards were added to plates and incubated for 2 hr at room temperature. Plates were washed and ErbB3-His was added for 1 hr at room temperature. Plates were washed and anti-His-HRP (Abeam, Cambridge, Mass.) was added for 1 hr at room temperature. Plates were developed using TMB and STOP solution and absorbance was read at 450 nM. PK parameters were analyzed using descriptive statistics including the median, mean and 95% confidence intervals around parameter estimates by dose level. All PK parameters included Cmax, Tmax, AUC (area under the concentration curve), clearance, volume of distribution at steady state (Vdss), and the terminal elimination half-life. Estimation of the PK parameters was performed using standard non-compartmental methods.

### Example 2

When patients with cancer are treated with a combination of an mTOR inhibitor (as exemplified by everolimus (Afinitor®) and INK-128 (alternate name: 3-(2-amino-5-benzoxazolyl)-1-(1-methylethyl)-1H-pyrazolo[3,4-d]pyrimidin-4-amine; CAS number: 1224844-38-5)) and MM-141, they (a) receive a therapeutic benefit that is equal to or greater than that of treatment with either mTOR inhibitor or MM-141 alone when each is administered as monotherapy at the same dose as in the combination, with fewer incidences of adverse events, or (b) receive a benefit that is greater than with either treatment alone when each is administered as monotherapy at the same dose as in the combination, which benefit occurs with an incidence of adverse events that is no higher than that for each of the individual treatments. Patients are dosed with MM-141, e.g., at 12 mg/kg weekly (q1w), 20 mg/kg q1w, or at 40 mg/kg every two weeks (q2w) by intravenous (IV) infusion and with an mTOR inhibitor (everolimus at 5 mg/kg or 10 mg/kg orally, once per day (qd) or INK128, e.g., at 7-40 mg orally qw, qd×3d qw, or qd×5d qw. MM-141 is administered at a 120 minute IV infusion for the first dose and if well tolerated, subsequent doses are 90 minute IV infusion at the frequency defined.

### Example 3

This Example discloses a method of treatment of patients with cancer with a combination of an mTOR inhibitor (for example, everolimus (Afinitor®) or INK-128) and an anti-estrogen therapy (for example exemestane, letrozole, anastrozole, fulvestrant and tamoxifen) and MM-141, wherein the therapeutic effect of the combination is larger than the therapeutic effect of an mTOR inhibitor or an anti-estrogen therapeutic or MM-141 alone when each is administered as monotherapy at the same dose as in the combination. MM-141 and mTOR inhibitors are dosed as specified in Example 2 and anti-estrogen therapies are dosed as per manufacturer's recommendation (exemestane at 25 mg orally, once per day; letrozole at 2.5 mg orally, once per day; anastrozole at 1 mg orally, once per day; fulvestrant at 250 mg or 500 mg intramuscularly on days 1, 15, 29 and once monthly thereafter; and tamoxifen at 10 mg or 20 mg orally, once or twice per day). MM-141 is administered as defined in Example 2.

### Example 4

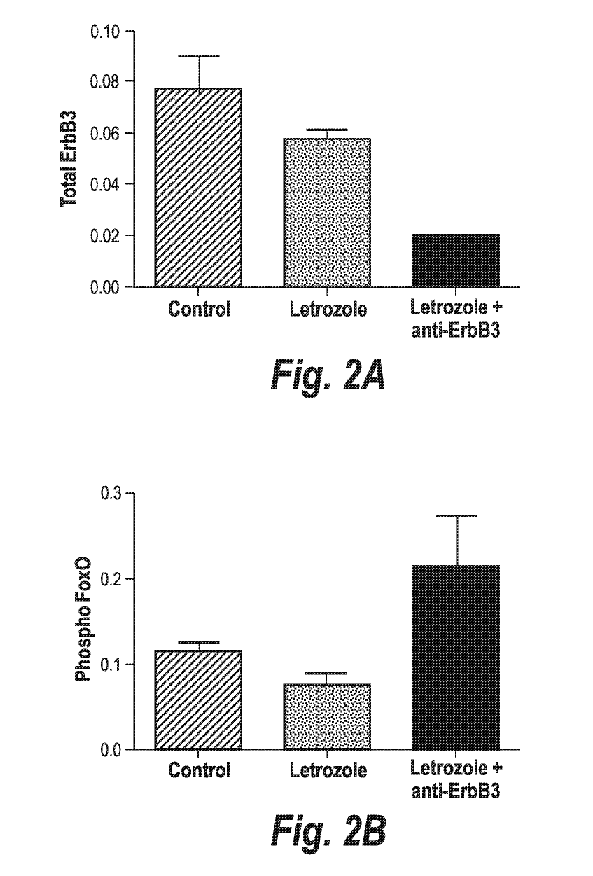

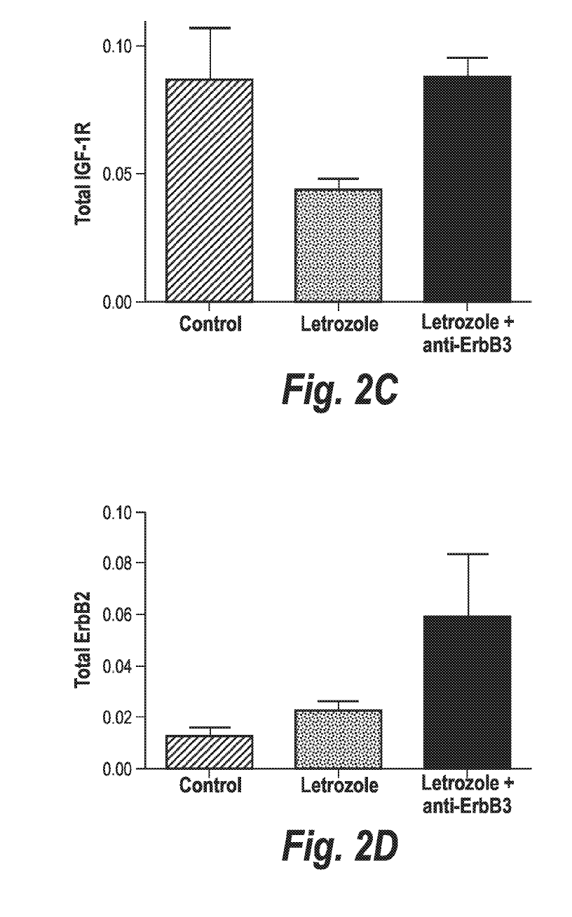

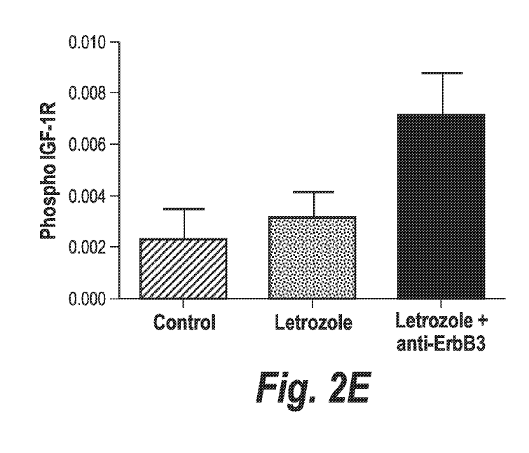

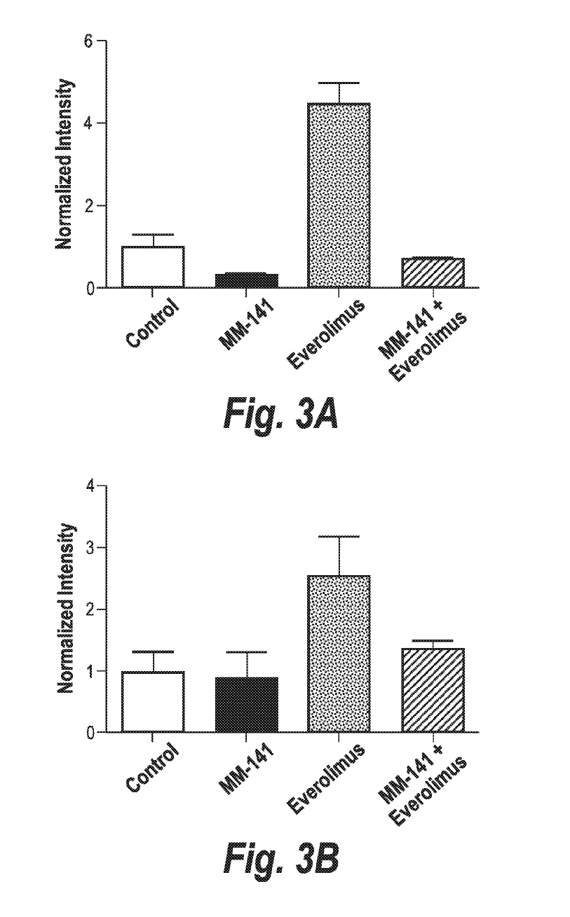

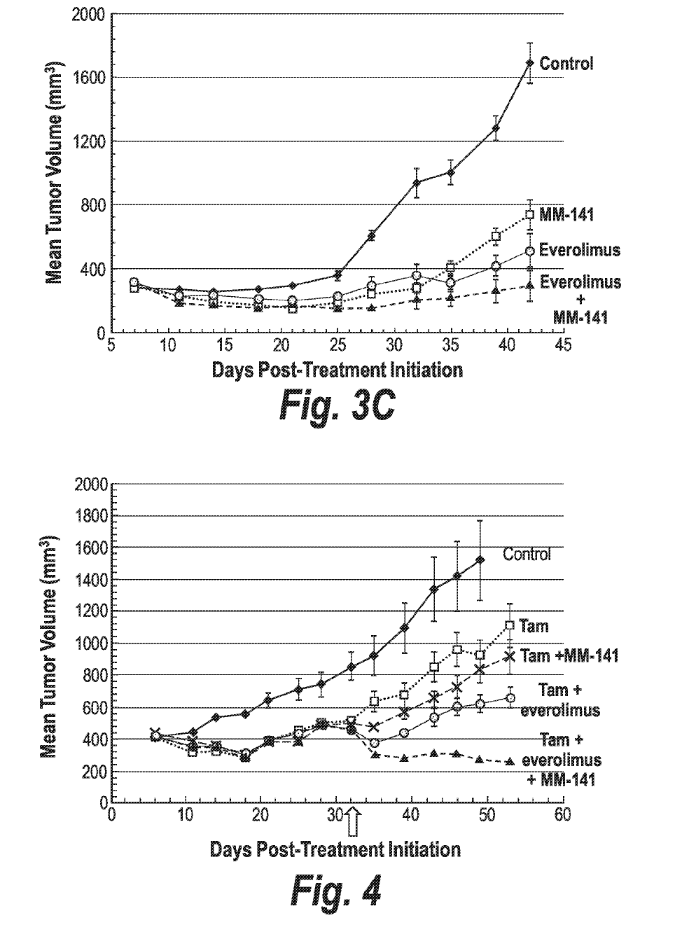

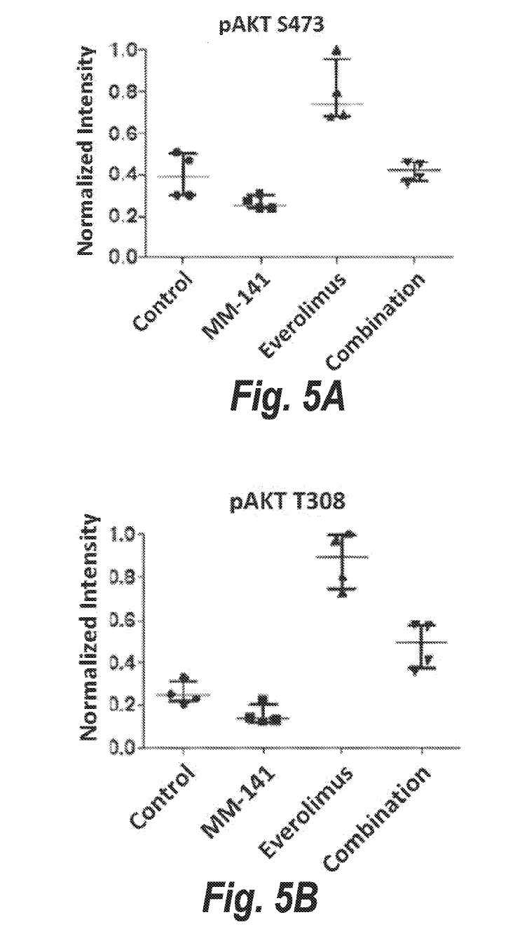

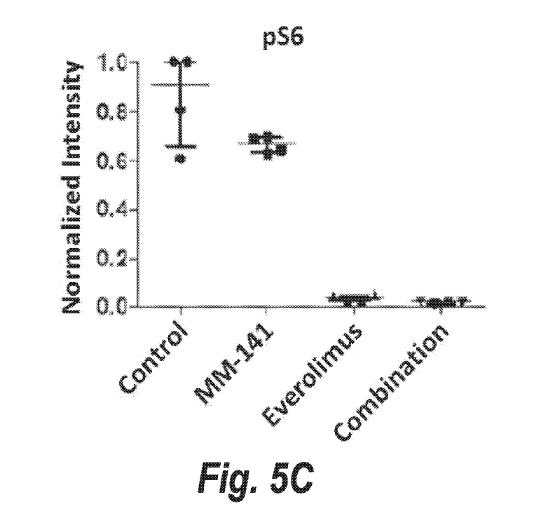

This Example demonstrates the advantages of combination therapy per Examples 2 and 3 in preclinical models. In preclinical mouse xenograft experiments conducted with MCF-7 breast cancer cell lines engineered to over-express the placental aromatase gene (MCF-7Ca; Jelovac et al., 2005 [Cancer Research, 65:5439]) we demonstrate that co-administration of the aromatase inhibitor letrozole and an anti-ErbB3 antibody leads to down regulation of ErbB3 (FIG. 2A) and triggers compensatory FoxO-dependent up-regulation of IGF-1R and ErbB2 (FIGS. 2B-D). This results in the activation of IGF-1R mediated signaling (FIG. 2E) resulting in upstream re-amplification of PI3K/AKT/mTOR signaling ultimately limiting the effectiveness of the letrozole and anti-ErbB3 antibody combination. In CAKI-1 renal cell carcinoma (RCC) cell line xenograft PD experiments, MM-141 treatment resulted in down regulation of IGF-1R and ErbB2. On the other hand, administration of everolimus resulted in upregulation of IGF-1R and ErbB2, and this resistance mechanism was inhibited by co-administration of MM-141 (FIG. 3A-B). The combination of MM-141 and everolimus was consistently more active than either single agent in suppressing xenograft tumor growth (FIGS. 3A-C). Additionally, in a BT-474-M3 breast cancer xenograft model, the co-administration of everolimus, MM-141 and tamoxifen had the most inhibitory effect on tumor growth compared to any of the agents alone following the development of resistance to tamoxifen (FIG. 4). Finally, in a pre-clinical PD study conducted in Ewing's sarcoma cell line xenografts (SK-ES-1), the combination of MM-141 and everolimus suppresses phospho-AKT (pAKT) and phospho-ribosomal S6 protein (pS6) activity more than everolimus alone (FIGS. 5A-C).

For the analyses described in FIGS. 2A-E, tumor xenografts were established by subcutaneous injection of 100 μL of a cell suspension consisting of 2.5×10⁷ MCF-7Ca cells, diluted 1:1 in Matrigel (BD Biosciences), into single sites on both flanks of recipient athymic ovariectomized female mice. As these mice were deficient in adrenal androgens, they were supplemented with daily subcutaneous injection of the aromatase substrate androstenedione (Δ4A; 100 μg/mouse/day; Sigma) for the duration of the experiment. Tumor formation was monitored weekly and tumor volumes were calculated following caliper measurement according to the formula (π/6*(length×width×width). Once the average measured tumor volume had reached 250-350 mm³, mice were randomized into groups of 10 and treatment was initiated. Overall, the average tumor volume per group was equivalent across all groups.

For injection, letrozole (Sigma) and MA were prepared in 0.3% hydroxypropylcellulose and anti-ErbB3 (Merrimack Pharmaceuticals) was diluted in 0.9% NaCl solution. Mice were treated by subcutaneous injection with letrozole (10 μg/mouse/d×5 days/week (qd×5)) or by intraperitoneal (i.p.) injection with anti-ErbB3 (750 μg/mouse, twice weekly) or vehicle (0.9% NaCl solution, twice weekly) as indicated. Treatments were continued for the duration of the study. Mice were euthanized at 24 h (Control) and at the end of the study (24 weeks; Letrozole and anti-ErbB3) respectively, and tumors were flash-frozen in liquid nitrogen following extraction. Lysates were generated and western blot analysis was performed.

For the PD study results shown in FIGS. 3A-B and FIGS. 5A-C, NOD/SCID female mice were inoculated with 12×10⁶ CAKI-1 or 10×10⁶ SK-ES-1 cells, respectively, in 1:1 Matrigel® suspension. Once xenograft tumors had formed and reached an average tumor volume of 300 mm³ (Day 0), mice (5/group) were dosed as follows. CAKI-1 xenograft mice were treated with 2 doses of MM-141 (30 mpk, i.p., Day 0 and Day 3), everolimus (10 mpk, orally, Day 2 and Day 3), or the combination of both at the same dose as described for the monotherapies. Tumors were harvested 24 hours after the second dose of drug; lysates were generated and subjected to western blot analysis.

For the efficacy study described in FIG. 3C, NOD/SCID female mice were inoculated with 8×10⁶ CAKI-1 cells. Once xenograft tumors had formed and reached an average tumor volume of 300 mm³, mice were dosed with MM-141 (25 mpk, i.p., q3d), everolimus (3 mpk, orally, qd), or the combination of both at the same dose as described for the monotherapies for the duration of the study (10 mice/group). Tumor volumes were measured weekly as outlined for FIGS. 2(A-E).

For the efficacy study outlined in FIG. 4, nu/nu female mice were implanted with 60 day, slow release estrogen pellets subcutaneously (SE-121, 0.72 mg; Innovative Research of America) the day before inoculating with 20×10⁶ BT-474-M3 cells subcutaneously. For tamoxifen treated mice, tamoxifen pellets (free base, 60 day release, 5 mg; Innovative Research of America) were implanted subcutaneously on day 7 post-inoculation. Following the re-growth of tamoxifen-treated tumors to >20% of their initial tumor volume (considered tamoxifen resistant; day 32 (open arrow)), mice were re-randomized to receive tamoxifen alone (5 mg pellet), or co-administered with MM-141 (30 mg/kg, i.p., q3d), everolimus (3 mpk, p.o., qd) or the combination of MM-141 and everolimus dosed as described. Mice were continuously dosed and tumor volumes measured as outlined for a further 21 days.

Taken together these results show that treatment with 2-way combinations of MM-141 and an mTOR inhibitor and 3-way combinations of MM-141, an mTOR inhibitor and an anti-estrogen agent provides markedly improved clinical activity compared to treatment with mTOR inhibitor monotherapy and to treatment with mTOR inhibitor/anti-estrogen combination therapy, respectively.

### Example 5

This Example discloses a method of treatment of patients with cancer with a combination of a nucleoside metabolic inhibitor (for example, gemcitabine (Gemzar®) or fluorouracil (5-FU)) and a taxane (for example, paclitaxel, docetaxel or nab-paclitaxel) and MM-141, wherein the therapeutic effect of the combination is larger than the therapeutic effect of any of the drugs alone when each is administered as monotherapy at the same dose as in the combination. MM-141 is dosed and administered as specified in Example 2, and the taxane and the nucleoside metabolic inhibitor are dosed and administered according to manufacturer's instructions. The nucleoside metabolic inhibitor and taxane are administered as IV infusions, e.g., over 40 minutes each on a 28 day cycle weekly for three weeks followed by one week off.

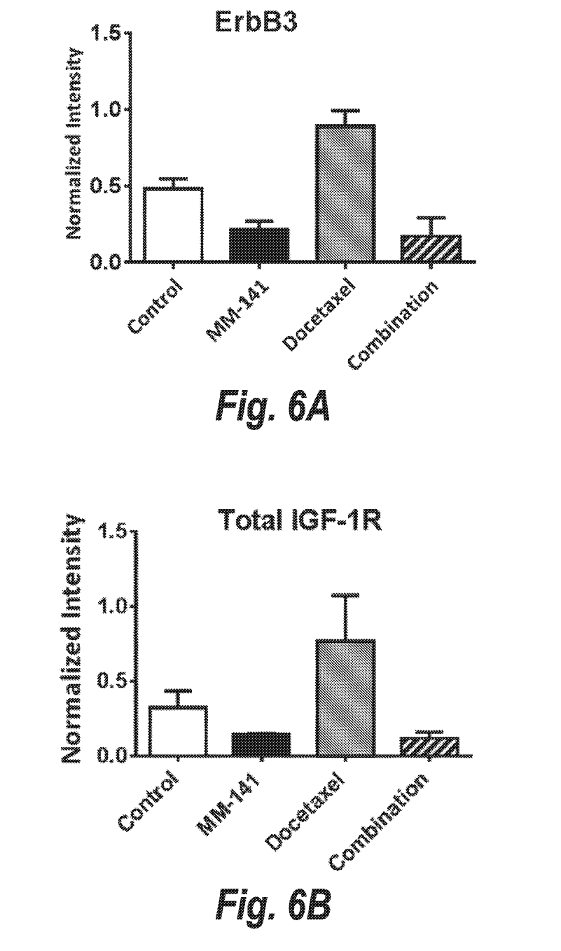

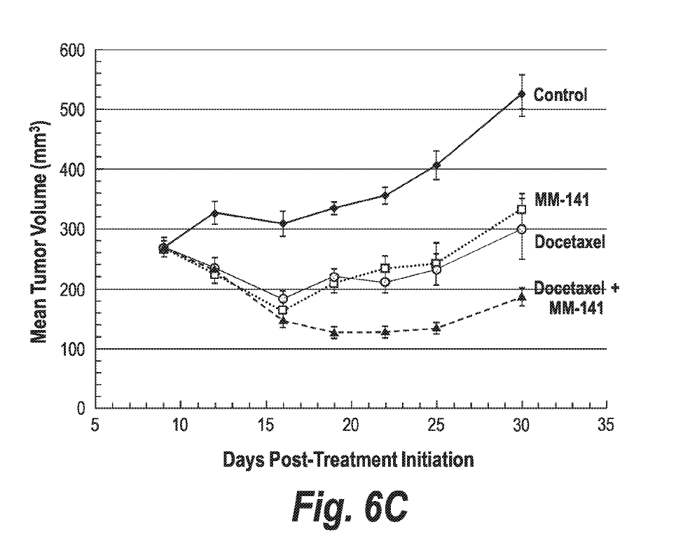

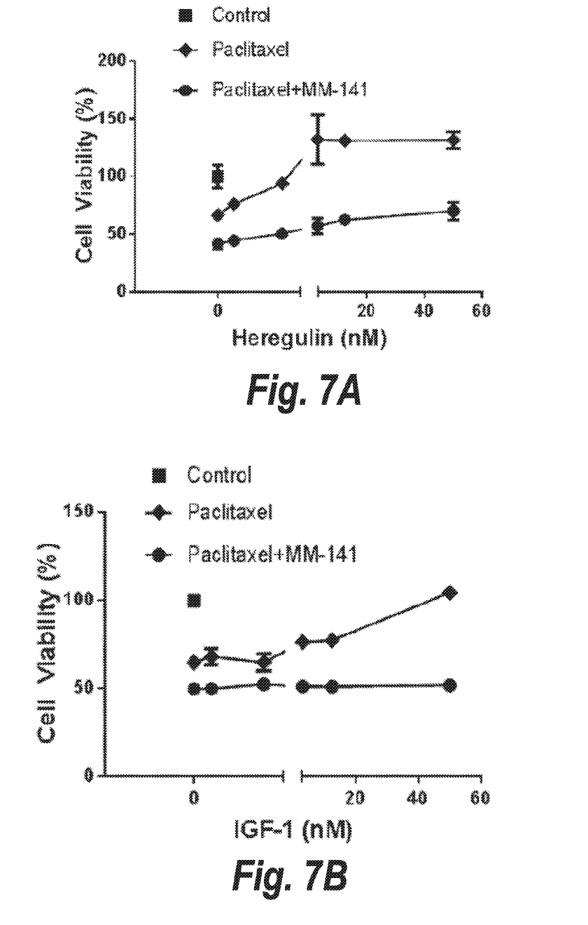

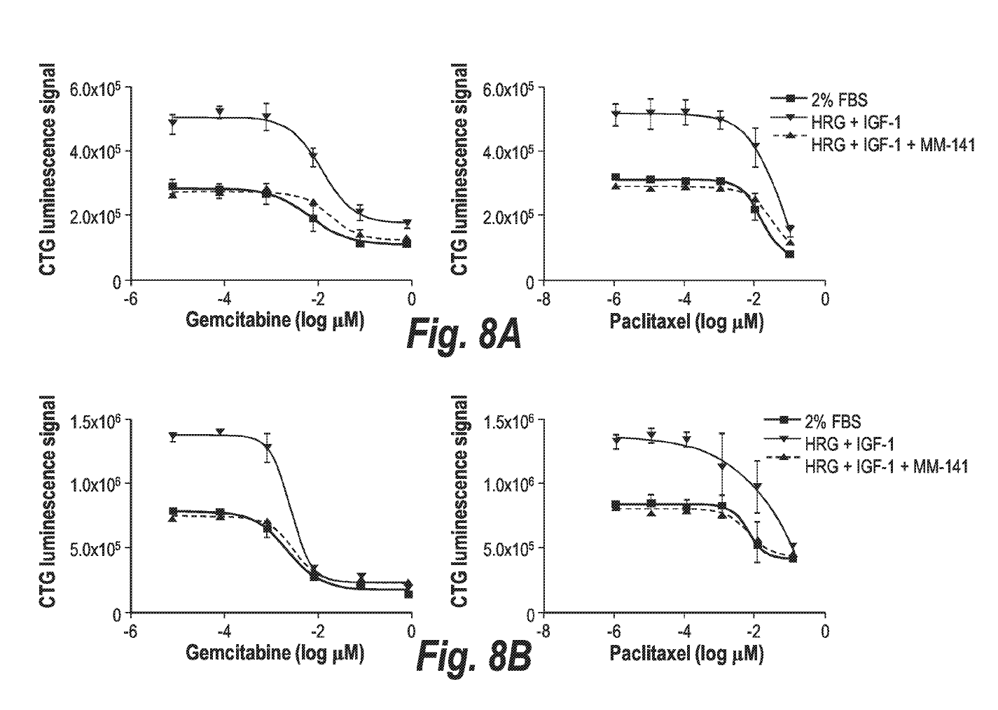

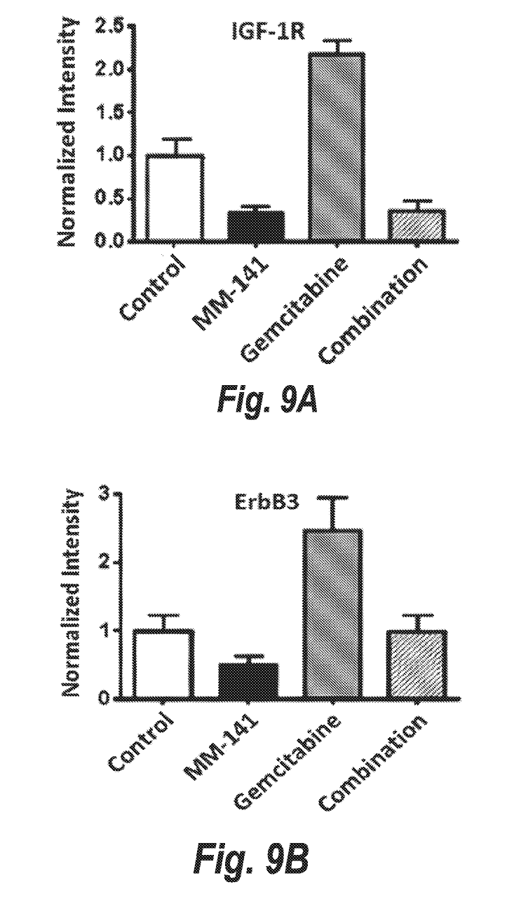

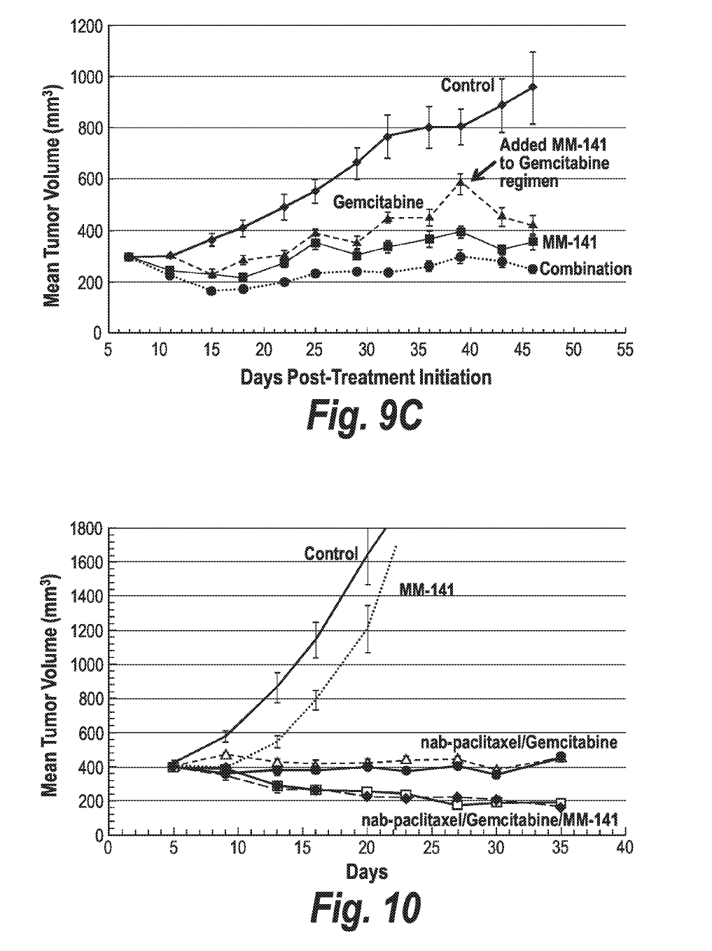

Preclinical experiments conducted with MM-141 have demonstrated the advantages of combining this regimen with docetaxel. In DU145 prostate cancer cells, docetaxel treatment up-regulated both IGF-1R and ErbB3 protein expression levels, two key receptor pathways involved in driving survival signaling through AKT. However, the up-regulation of these receptors was inhibited by combining docetaxel treatment with MM-141 (FIGS. 6A, B). Furthermore, the combination of MM-141 with docetaxel was more active in inhibiting the growth of DU145 derived xenograft tumors than either single agent alone (FIG. 6C). Further, administration of either IGF-1 or heregulin abrogated the cytotoxic effect of paclitaxel on three-dimensional cultures of BxPC-3 (KRAS wild-type) pancreatic cancer cells and this resistance was reversed by addition of MM-141 (FIGS. 7A-B). Moreover, administration of a combination of IGF-1 and HRG dampened the cytotoxic effect of gemcitabine and paclitaxel on two-dimensional cultures of BxPC-3 and CFPAC-1 (KRAS mutant) pancreatic cancer cells and this resistance was reversed by the addition of MM-141 (FIGS. 8A-B). MM-141 also showed therapeutic synergy with gemcitabine in BxPC-3 cell line xenografts (FIGS. 9A-C). The administration of gemcitabine to mice led to IGF-1R and ErbB3 up-regulation and activation. Co-administration of MM-141 reversed the receptor up-regulation and increased the activity of gemcitabine in the combination arm. Following the development of resistance to gemcitabine, the addition of MM-141 to the gemcitabine regimen led to decreased tumor growth.

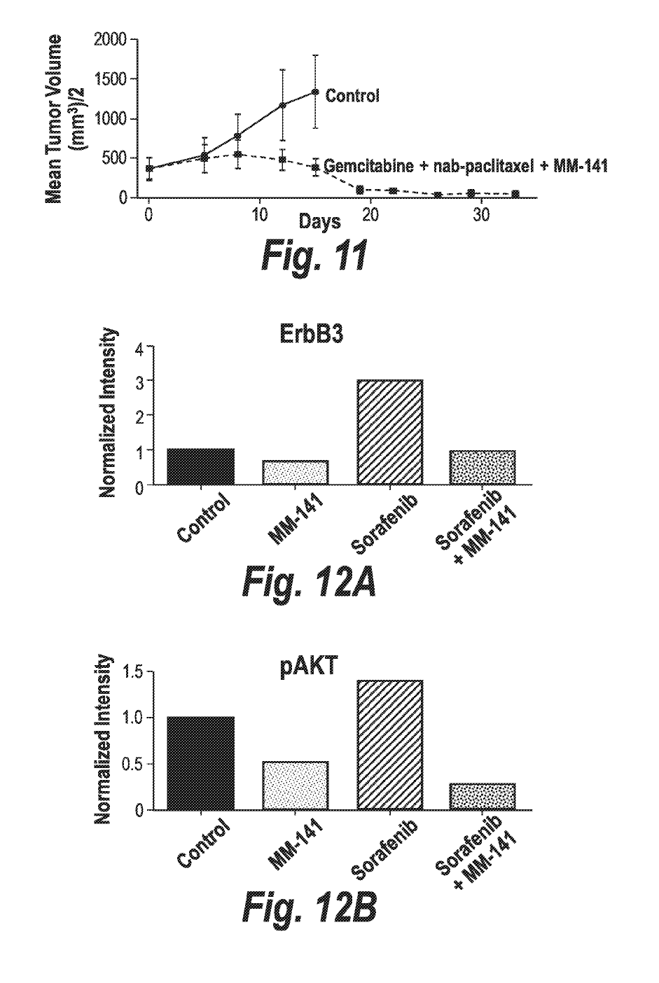

Additional preclinical studies have indicated the advantages of adding MM-141 in combination with a regimen comprising both a taxane and gemcitabine. In HPAF-II (KRAS mutant) pancreatic cancer cells, treatment with a triple combination regimen comprising nab-paclitaxel/gemcitabine/MM-141 had a sustained inhibitory effect on tumor growth compared to a combination of nab-paclitaxel/gemcitabine at equivalent concentration (FIG. 10). Moreover, in a patient-derived primary pancreatic xenograft tumor model, a regimen comprising nab-paclitaxel/gemcitabine/MM-141 was highly active in inhibiting tumor growth (FIG. 11). Taken together, these data indicate synergistic effects of MM-141, gemcitabine and taxanes supporting its clinical development in inoperable metastatic pancreatic cancer.

For the PD studies described in FIGS. 6A-B and FIGS. 9 A-B, nu/nu female mice were inoculated with 8×10⁶ DU145 or 5×10⁶ BxPC-3 cells, respectively, in 1:1 Matrigel® suspension. Once xenograft tumors had formed and reached an average tumor volume of 300 mm³ (Day 0), mice (4/group) were dosed accordingly. DU145 xenograft mice were treated with 2 doses of MM-141 (30 mpk, i.p., Day 0 and Day 3), a single dose of docetaxel (20 mpk, i.p., Day 3), or the combination of both MM-141 and docetaxel as dosed for the monotherapy. Tumors were harvested 24 hours after the second dose of MM-141 (Day 4). BxPC-3 xenografted mice were treated with 3 doses of MM-141 (30 mpk, i.p., Day 0, 3, 6), 2 doses of gemcitabine (150 mpk, i.p., Day 0, 6), or the combination of both MM-141 and gemcitabine as dosed for the monotherapy. Tumors were harvested 24 hours after the third dose of MM-141 (Day 7). Tumor lysates were generated and subjected to western blot analysis.

For the efficacy studies (FIGS. 6C and 9C), nu/nu female mice were inoculated with 8×10⁶ DU145 cells and 5×10⁶ BxPC-3 cells, respectively. Once xenograft tumors had formed and reached an average tumor volume of 275-300 mm³, dosing was initiated. DU145 xenografted mice (10/group) were dosed with MM-141 (30 mpk, i.p., q3d), docetaxel (10 mpk, i.p., q7d), or the combination of MM-141 and docetaxel as dosed for the monotherapy for the duration of the study. BxPC-3 xenografted mice (10/group) were dosed with MM-141 (30 mpk, i.p., q3d), gemcitabine (150 mpk, i.p., q6d), or the combination of MM-141 and gemcitabine as dosed for the monotherapy. Following the development of resistance to gemcitabine alone, MM-141 (30 mpk, i.p., q3d) was added to the gemcitabine regimen on day 41. Tumor volume was measured weekly as described in Example 4.

The cell viability assay (FIGS. 7A-B and 8A-B) was carried out using a CellTiter-Glo® (CTG) assay (Promega), which determines the number of viable cells in a culture based on quantitation of the ATP present. In FIGS. 7A-B, BxPC-3 cells were grown in low serum alone or with exogenous IGF-1 or HRG added (0-50 nM). Cell proliferation was measured following treatment with various concentrations of both ligands in the presence of paclitaxel (50 nM), either alone or in combination with MM-141 (500 nM). For the cell viability assays described in FIGS. 8(A-B), (FIG. 8A) BxPC-3 and (FIG. 8B) CFPAC-1 cells were cultured in low (2%) serum alone or with a combination of IGF-1 (50 nM) and HRG (10 nM). Cell proliferation was measured following treatment with various concentrations of gemcitabine (10 pM-1 μM) or paclitaxel (1 pM-100 nM), either alone or in combination with MM-141 (500 nM).

For the efficacy study described in FIG. 10, SCID beige female mice were inoculated with 5×10⁶ HPAF-II cells. Once xenograft tumors had formed and reached an average tumor volume of 400 mm³, dosing was initiated. HPAF-II xenografted mice (10/group) were administered PBS as control (q3d, i.p.; solid black line), MM-141 as a monotherapy (30 mg/kg, q3d, i.p.; dotted black line), a combination of nab-paclitaxel (5 mg/kg, q3d, i.p.) and gemcitabine (40 mg/kg, q6d, i.p.) (open triangles, dashed black line), a combination of nab-paclitaxel (10 mg/kg, q3d, i.p.) and gemcitabine (40 mg/kg, q6d, i.p.) (solid black circles, solid black line), a triple combination of nab-paclitaxel (5 mg/kg, q3d, i.p.), gemcitabine (40 mg/kg, q6d, i.p.) and MM-141 (30 mg/kg, q3d, i.p.; grey squares, solid black line), or a triple combination of nab-paclitaxel (10 mg/kg, q3d, i.p.), gemcitabine (40 mg/kg, q6d, i.p.) and MM-141 (30 mg/kg, q3d, i.p.; solid diamonds, dashed black line). Tumor volume was measured twice weekly as described in Example 4, and error bars shown represent standard error of the mean per group.

For the efficacy study outlined in FIG. 11, 2-3 mm³ chunks of xenografted patient-derived pancreatic tumor were implanted subcutaneously into the right flank of SCID male mice. Once xenograft tumors had formed and reached an average tumor volume of 400 mm³, dosing was initiated. Mice (10/group) were administered PBS as control (q3d, i.p.; black circles, solid black line) or a combination of nab-paclitaxel (30 mg/kg, q7d, i.v.), gemcitabine (50 mg/kg, twice weekly, i.p.) and MM-141 (30 mg/kg, q3d, i.p.; solid squares, dashed black line). Tumor volume was measured twice weekly.

### Example 6

This Example discloses a method of treatment of patients with sorafenib (Nexavar®)-resistant hepatocellular carcinoma (HCC) with MM-141 monotherapy. Sorafenib-resistant patients are those who have progressed while on sorafenib treatment. Patients are dosed with MM-141 at 20 mg/kg q1w or 40 mg/kg q2w by IV infusion. MM-141 is administered as outlined in Example 2. As described in Examples 2 and 3, MM-141 can block acquired resistance to everolimus, indicating a benefit of MM-141 in HCC. In addition, in the HCC cell line HepG2, ErbB3 and pAKT levels were up-regulated in response to sorafenib treatment, which was overcome by addition of MM-141 (FIGS. 12A-B).

HepG2 cells were plated on 12 well dishes (3×10⁵ cells per well) in 10% serum-containing media and incubated overnight. Once the cell density had reached approximately 70%, sorafenib (5 μM) was added alone or in combination with MM-141 (500 nM) for 2 hours. Following treatment, cells were harvested in lysis buffer containing protease and phosphatase inhibitors and analyzed by western blotting.

### Example 7

Cancer patients (pancreatic cancer, ovarian cancer, sorafenib-naive or sorafenib-refractory hepatocellular carcinoma, parathyroid cancer, sarcoma, lung cancer or breast cancer) are treated with a combination of an anthracycline (e.g., doxorubicin, epirubicin, or Doxil®) and MM-141. MM-141 is dosed and administered as specified in Example 2 and the anthracycline is dosed and administered as per manufacturer's instructions. The therapeutic effect of the combination will be larger than the therapeutic effect of the anthracycline or MM-141 alone when each is administered as monotherapy at the same dose as in the combination.

### Example 8

This Example discloses a method of treatment of patients with cancer with a combination of a taxane (for example paclitaxel, docetaxel, or nab-paclitaxel) and MM-141, wherein the therapeutic effect of the combination is larger than the therapeutic effect of a taxane or MM-141 alone when each is administered as monotherapy at the same dose as in the combination. MM-141 is dosed and administered as specified in Example 2 and the taxane is dosed and administered as per manufacturer's instructions.

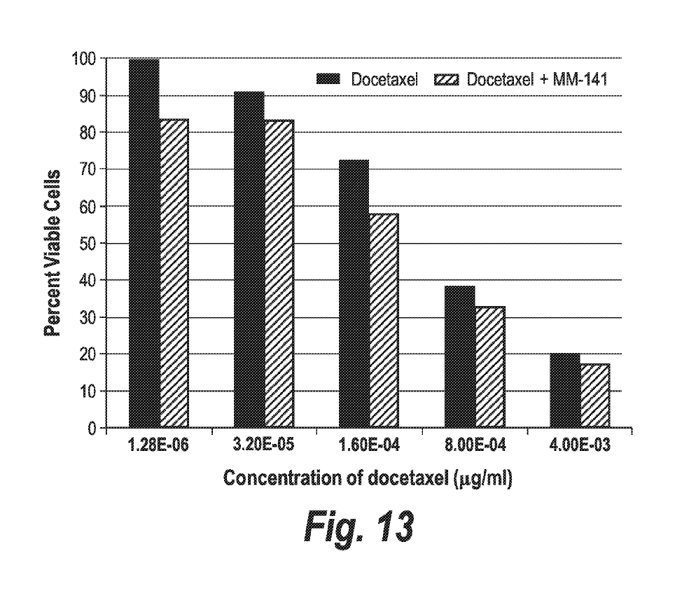

As indicated in FIGS. 5A-C, dosing with MM-141 in combination with docetaxel was more active at inhibiting the growth of DU145 prostate cancer cell line xenografts than dosing with either agent alone. Consistently, in vitro experiments evaluating the cytotoxic effects of MM-141 in combination with docetaxel demonstrated activity over a wide range of docetaxel concentrations (FIG. 13). In brief, BxPC-3 cell viability was measured following treatment with various doses of docetaxel (4 ng/mL-1.28 pg/mL) added, alone or in combination with MM-141 (500 nM) using a CTG assay.

### Example 9

Cancer patients (pancreatic cancer, ovarian cancer, sorafenib-naive or sorafenib-refractory hepatocellular carcinoma, parathyroid cancer, sarcoma, lung cancer and or breast cancer) are treated with a combination of a phosphoinositide 3 kinase (PI3K) inhibitor (e.g., BKM120 (alternate name: (5-[2,6-Di(4-morpholinyl)-4-pyrimidinyl]-4-(trifluoromethyl)-2-pyridinamine; CAS number: 944396-07-0), GDC-0941 (alternate name: 2-(1H-Indazol-4-yl)-6-[[4-(methylsulfonyl)-1-piperazinyl]methyl]-4-(4-morpholinyl)-thieno[3,2-d]pyrimidine; CAS number: 957054-30-7), PX-866 (CAS number: 502632-66-8), GDC-0032 (alternate name: 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide; CAS number: 1282512-48-4), BYL719 (alternate name: (2S)—N1-[4-methyl-5-[2-(2,2,2-trifluoro-1,1-dimethylethyl)-4-pyridinyl]-2-thiazolyl]-1,2-pyrrolidinedicarboxamide; CAS number: 1217486-61-7), INK1117 (Millenium/Intellikine), GSK2636771 (alternate name: 2-Methyl-1-(2-methyl-3-(trifluoromethyl)benzyl)-6-morpholino-1H-benzo[d]imidazole-4-carboxylic acid; CAS number: 1372540-25-4), TGX-221 (alternate name: (±)-7-Methyl-2-(morpholin-4-yl)-9-(1-phenylaminoethyl)-pyrido[1,2-a]-pyrimidin-4-one; CAS number: 663619-89-4), GS-1101 (alternate name: (S)-2-(1-(9H-purin-6-ylamino)propyl)-5-fluoro-3-phenylquinazolin-4(3H)-one; CAS number: 870281-82-6), or IPI-145 (alternate name: (S)-3-(1-((9H-purin-6-yl)amino)ethyl)-8-chloro-2-phenylisoquinolin-1(2H)-one; CAS number: 1201438-56-3)) and MM-141. MM-141 is dosed and administered as specified in Example 2 and the PI3K inhibitor is dosed and administered as per manufacturer's instructions. The therapeutic effect of the combination will be larger than the therapeutic effect of the PI3K inhibitor or MM-141 alone when each is administered as monotherapy at the same dose as in the combination.

### Example 10

This Example discloses co-administration of MM-141 with a PI3K inhibitor and an anti-estrogen therapy as a method of treatment of patients with cancer. MM-141 and the PI3K inhibitor are co-administered as described in Example 9. Anti-estrogen therapy (for example exemestane, letrozole, anastrozole, fulvestrant and Tamoxifen) will be dosed and administered as per Example 3.

### Example 11

This Example discloses co-administration of a MEK1 and/or MEK2 inhibitor (e.g., trametinib, BAY 86-9766) and MM-141 as a method of treatment of patients with cancer, in which the therapeutic effect of the combination is larger than the therapeutic effect of the MEK1 and/or MEK2 inhibitor or MM-141 alone when each is administered as monotherapy at the same dose as in the combination. MM-141 is dosed and administered as specified in Example 2 and the MEK1 and/or MEK2 inhibitor is dosed and administered as per manufacturer's instructions.

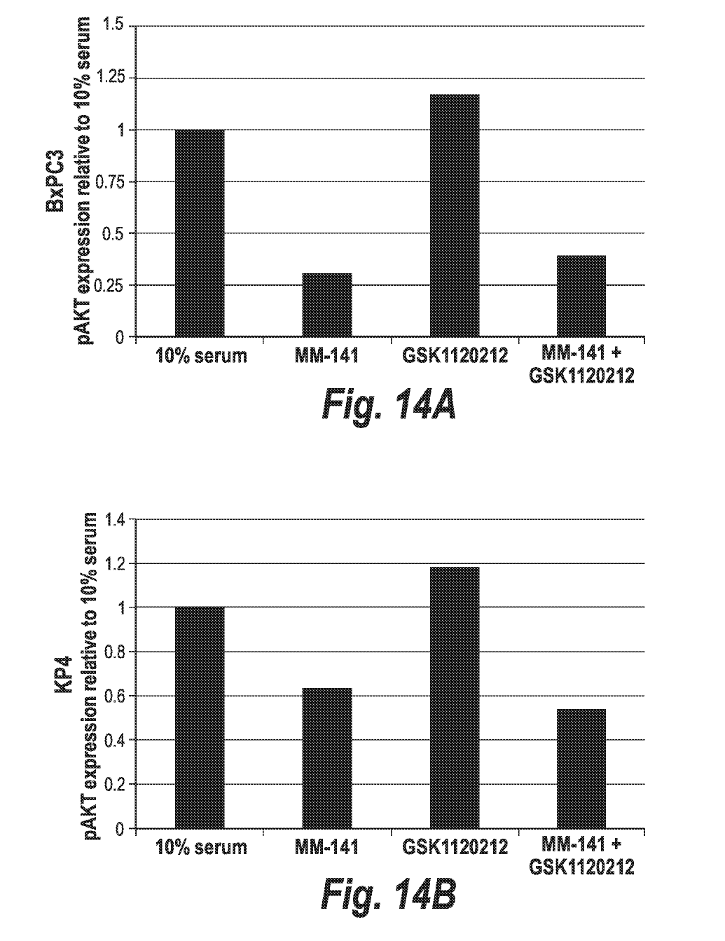

Up-regulation of pAKT as a potential feedback survival mechanism has been shown in GSK-1120212 treated BxPC-3 (KRas wild-type) and KP4 (KRas mutant) pancreatic cancer cells. MM-141 down-regulates the basal level of pAKT in BxPC-3 and KP4 cells, and also decreases the pAKT upregulation induced by the MEK inhibitor (FIGS. 14A-B), indicating the benefit of combining both therapies. BxPC-3 and KP4 cells were treated for 24 h in vitro with GSK-1120212 (250 nM), MM-141 (1000 nM) or the combination of both MM-141 and GSK-1120212 as dosed for the monotherapies. Following treatment, cells were harvested in cell lysis buffer and western blotting was performed and quantified for pAKT (Serine 473).

### Example 12

Cancer patients (pancreatic cancer, ovarian cancer, sorafenib-naive or sorafenib-refractory hepatocellular carcinoma, parathyroid cancer, sarcoma, lung cancer and or breast cancer) are treated with a combination of an anaplastic lymphoma kinase (ALK) inhibitor (e.g., alectinib or crizotinib) and MM-141. MM-141 is dosed and administered as specified in Example 2 and the ALK inhibitor is dosed and administered as per manufacturer's instructions. The therapeutic effect of the combination will be larger than the therapeutic effect of the ALK inhibitor or MM-141 alone when each is administered as monotherapy at the same dose as in the combination.

### Example 13

This Example discloses a method of treatment of patients with cancer with a combination of a v-Raf murine sarcoma viral oncogene homolog B1 (BRAF) inhibitor (e.g., sorafenib, vemurafenib or dabrafenib) and MM-141, wherein the therapeutic effect of the combination is larger than the therapeutic effect of an BRAF inhibitor or MM-141 alone when each is administered as monotherapy at the same dose as in the combination. MM-141 is dosed and administered as specified in Example 2 and the BRAF inhibitor is dosed and administered as per manufacturer's instructions.

Pre-clinical research performed at Merrimack has already shown upregulation of ErbB3 activity in response to sorafenib treatment in the HCC cell line HepG2, which was overcome by addition of MM-141 (FIGS. 12A-B).

### Example 14

This Example discloses a method of treatment of patients with cancer (wherein the cancer is selected from the group consisting of pancreatic cancer, ovarian cancer (including high-grade serous ovarian cancer), sorafenib-naïve or sorafenib-refractory hepatocellular carcinoma, parathyroid cancer, sarcoma, lung cancer and breast cancer) with the combination therapies described in examples 2-13, wherein the patients have high levels of free IGF-1 in serum.

Retrospective analyses performed on patients treated with IGF-1R inhibitors have demonstrated the importance of measuring pre-treatment levels of circulating IGF-1.

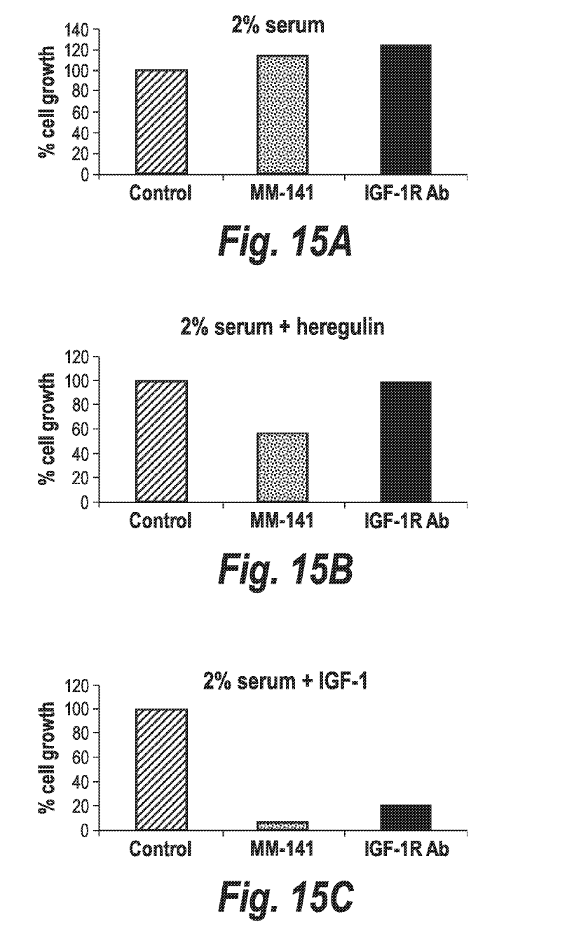

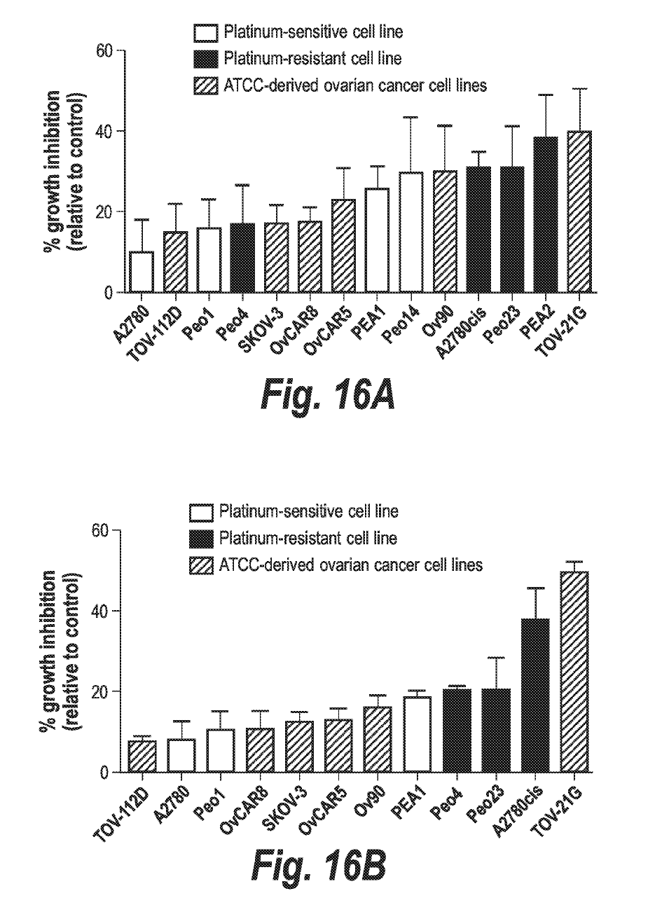

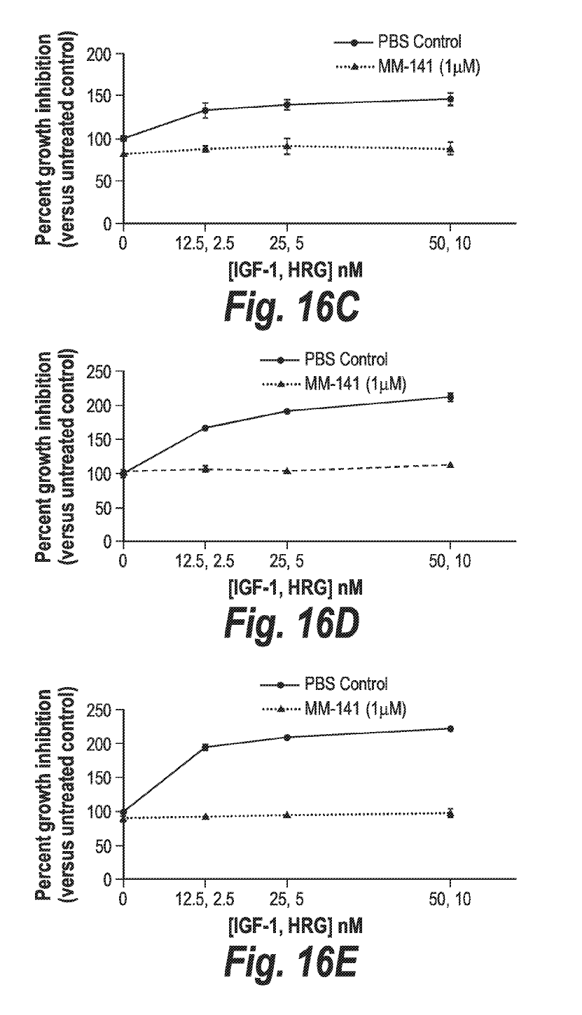

Preclinical data obtained using a pancreatic adenocarcinoma cell line (BxPC-3) demonstrate that MM-141 has optimal activity in the presence of IGF-1 (FIGS. 15A-C). BxPC-3 cells were grown in low (2%) serum condition, mimicking the effect of growth factor deprivation and a cellular proliferation assay was used to compare inhibition of cell growth following treatment with MM-141 or the single anti-IGF-1R component of MM-141. In the absence of exogenous ligand MM-141 was unable to inhibit cell growth, similar to the anti-IGF-1R antibody. Upon the addition of IGF-1 to the media, the anti-IGF-1R antibody was able to significantly inhibit cell growth, with MM-141 displaying maximal activity. BxPC-3 cells were also used to demonstrate the loss of sensitivity to paclitaxel in the presence of increasing concentrations of IGF-1 (FIGS. 7A-B). The addition of MM-141 to paclitaxel was able to re-sensitize cells to the cytotoxic properties of paclitaxel in a high IGF-1 setting.

### Example 15

This Example discloses a method of treatment of patients with cancer (wherein the cancer is selected from the group consisting of pancreatic cancer, ovarian cancer, sorafenib-naïve or sorafenib-refractory hepatocellular carcinoma, parathyroid cancer, sarcoma, lung cancer and breast cancer) with the combination therapies described in examples 2-13, wherein the patients have high levels of heregulin (HRG) in tissue.

Pre-clinically, pancreatic adenocarcinoma BxPC-3 cells were used to demonstrate that in presence of HRG, MM-141 was able to inhibit cell growth approximately 50% greater than in the absence of ligand (FIGS. 15A-C). Additionally, these cells also reflected a loss of sensitivity to paclitaxel in the presence of increasing concentrations of HRG (FIGS. 7A-B). The addition of MM-141 to paclitaxel was able to re-sensitize cells to the cytotoxic properties of paclitaxel in the presence of high HRG.

### Example 16

This Example provides actual clinical administration parameters (including dosage and administration) and preliminary results for an ongoing MM-141 phase 1 clinical trial treating tumors in human cancer patients.

Methods:

This is a Phase 1 dose-escalation study evaluating safety, tolerability, pharmacokinetic (PK), and pharmacodynamic (PD) properties of MM-141 as monotherapy (Arm A, n=15) and in combination with everolimus (Arm B) or with nab-paclitaxel and gemcitabine (Arm C, n=11).

TABLE 1
Clinical Trial Design

| | | | Arm B | | Arm C |
|---|---|---|---|---|---|
| | Arm A | | MM-141 + | | MM-141^{a} + |
| | MM-141^{a} | | everolimus^{b} + | | nab- |
| | Mono- | | anti-estrogen | | paclitaxel^{c} + |
| Cohort | therapy | Cohort | (optional) | Cohort | gemcitabine^{c} |
| 1A | 6 QW | 1B | X | 1C | X |
| 2A | 12 QW | 2B | X | 2C | X |
| 3A | 20 QW | 3B | X | 3C | X |
| 4A | 40 Q2W | 4B | 20 Q2W + 5 | 4C | 12 QW + |
| | | | | | 125 + 1000 |
| 4D | 20 QW | 5B | 40 Q2W + 5 | 5C | 20 QW + |
| | | | | | 125 + 1000 |
| | | 6B | MTD + 10 | | |

Cohort A: solid tumors
Cohort B: ER/PR+ breast cancer
Cohort C: pancreatic cancer
Cohort D: hepatocellular carcinoma
^{a}dosage is in mg/kg
^{b}dosage is in mg
^{c}dosage is in mg/m²

Three HCC patients in the Arm A 4D expansion cohort received MM-141 as a monotherapy at a weekly dose of 20 mg/kg. These patients underwent mandatory pre-treatment and optional post-treatment biopsies. Patients in the dose-escalation portion of Arm C received MM-141 at a weekly dose of 12 or 20 mg/kg in combination with weekly nab-paclitaxel (125 mg/m²) and gemcitabine (1000 mg/m²) (3 weeks on, 1 week off). Enrollment in Arm B (MM-141 in combination with everolimus) is ongoing.

Key inclusion criteria include cytologically or histologically confirmed advanced malignant solid tumors for which no curative therapy exists that has recurred or progressed following standard therapy; a body mass index between 18 and 32.5; measurable disease according to RECIST v1.1; and no insulin-dependent or uncontrolled diabetes.

Key primary and secondary objectives include determination of the maximum tolerated dose or recommended Phase 2 dose of MM-141 as a single agent, in combination with everolimus, and in combination with nab-paclitaxel and gemcitabine based on the safety, tolerability, PK, and PD; determination of the adverse event profile; and determination of the pharmacokinetic and immunogenicity parameters.

This study features a standard “3+3 design followed by additional expansion cohorts and combination arms. MM-141 is dosed weekly or bi-weekly for four week cycles. There is a four week dose-limiting toxicity (DLT) evaluation period prior to escalating to the next cohort.

Key study requirements are that patients are tested for free serum IGF-1 at screening; cohort 4D comprises mandatory pre-treatment biopsies and optional post-treatment biopsies; treatment arm B includes mandatory pre-treatment biopsies and mandatory post-treatment biopsies; patients are scanned every eight weeks; and the patients participate in daily glucose monitoring.

Preliminary Results:

Fifteen patients with advanced solid tumors were enrolled into the dose escalation portion of Arm A. No DLTs were observed at any of the studied dose levels. The safety, tolerability, PK and PD profile support weekly and bi-weekly MM-141 dosing. The Arm A expansion cohort 4D enrolled 3 patients with sorafenib-refractory HCC. The analysis of pre- and post-treatment biopsies confirmed that IGF-1R and ErbB3 are expressed in patients previously exposed to sorafenib, and their levels are decreased after MM-141 exposure. Eleven patients with advanced solid tumors were enrolled into Arm C, combining MM-141 with nab-paclitaxel and gemcitabine. One DLT of grade 3 abdominal cramping was seen at the MM-141 dose of 20 mg/kg weekly. An additional 3 patients were enrolled at that dose level and no further DLTs were seen.

### Example 17

This Example discloses a method of treatment of patients with platinum-sensitive and platinum-resistant ovarian cancer with a combination of a taxane (for example, paclitaxel, docetaxel or nab-paclitaxel) and MM-141, wherein the therapeutic effect of the combination is larger than the therapeutic effect of any of the drugs alone when each is administered as monotherapy at the same dose as in the combination. Patients are dosed with MM-141, e.g., at 12 mg/kg weekly (q1w), 20 mg/kg q1w, or at 40 mg/kg every two weeks (q2w) by intravenous (IV) infusion. MM-141 is administered at a 120 minute IV infusion for the first dose and, if the first dose is well tolerated, subsequent doses are 90 minute IV infusions at the frequency indicated above. The taxane is dosed according to manufacturer's instructions and administered as IV infusions, e.g., over 40 minutes each on a 28 day cycle weekly for three weeks followed by one week off.

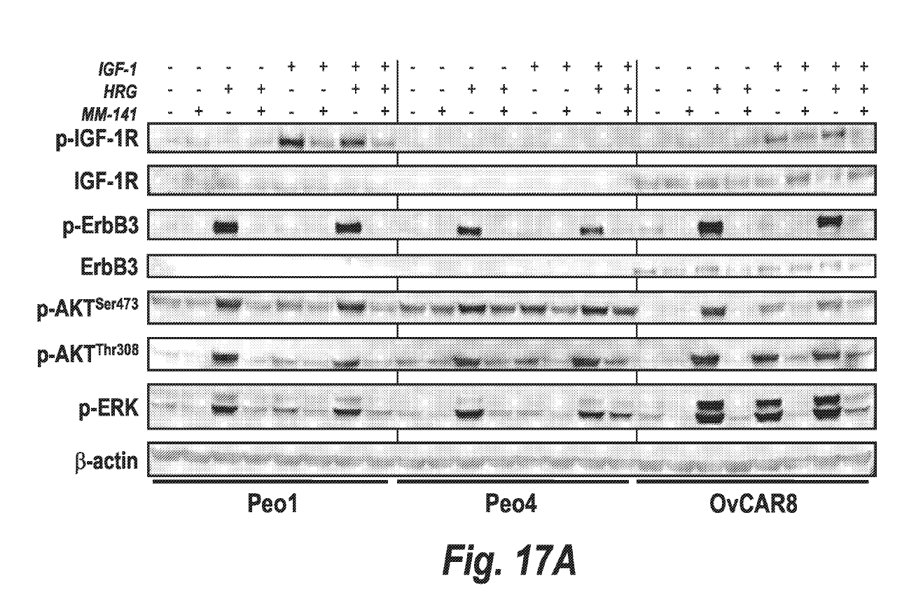

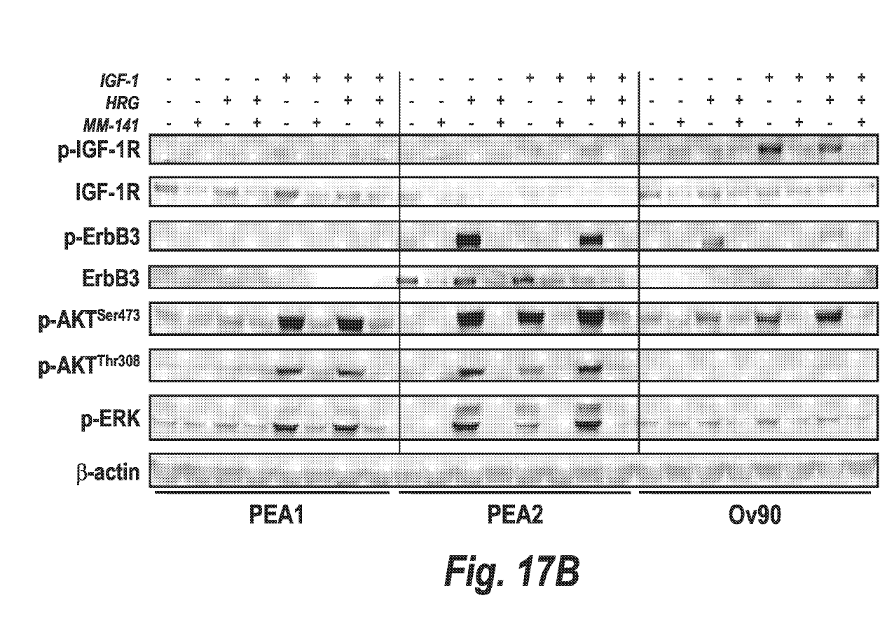

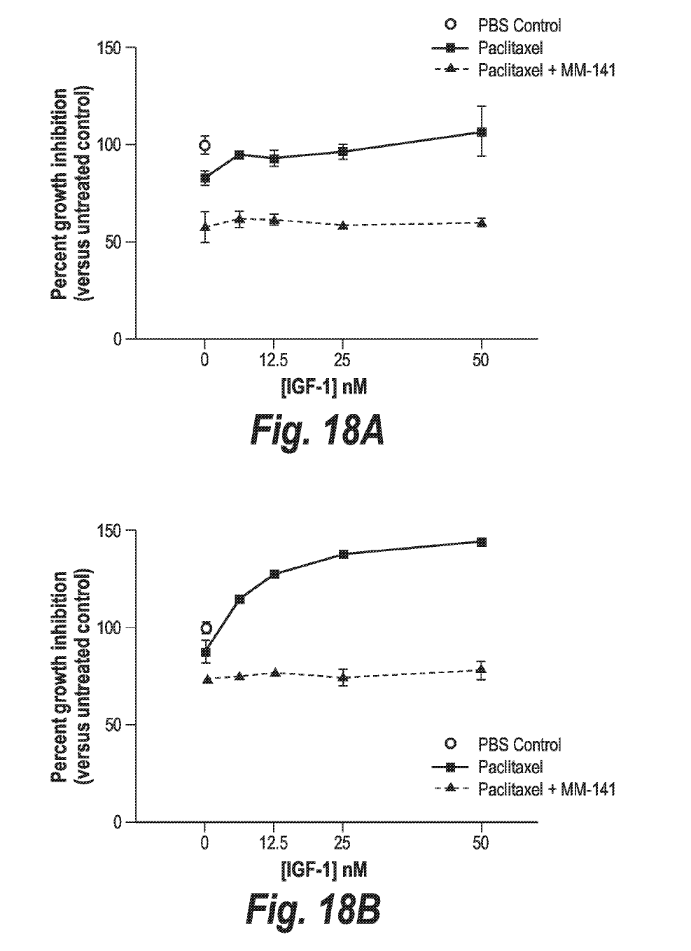

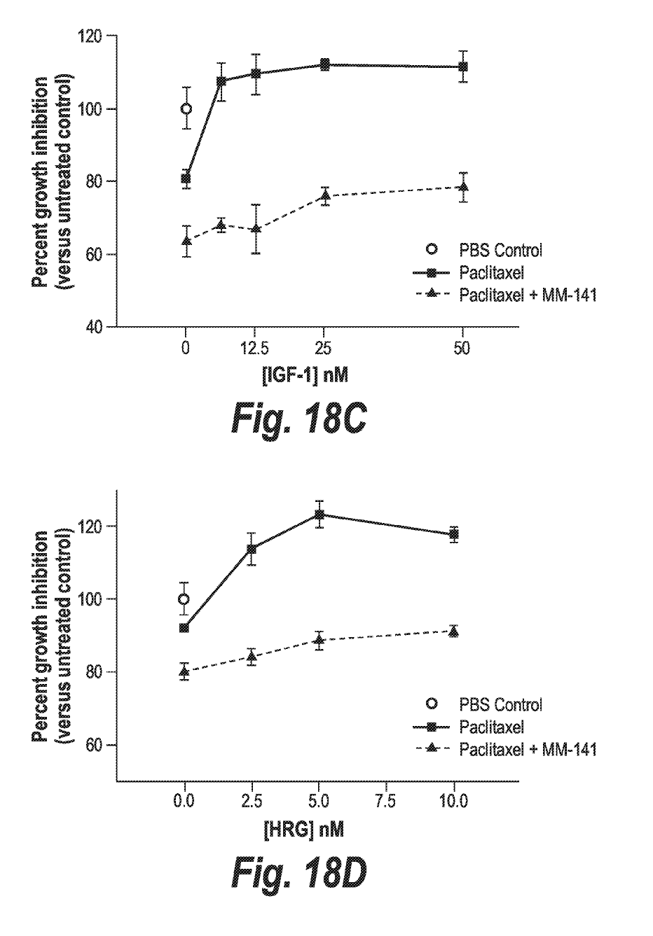

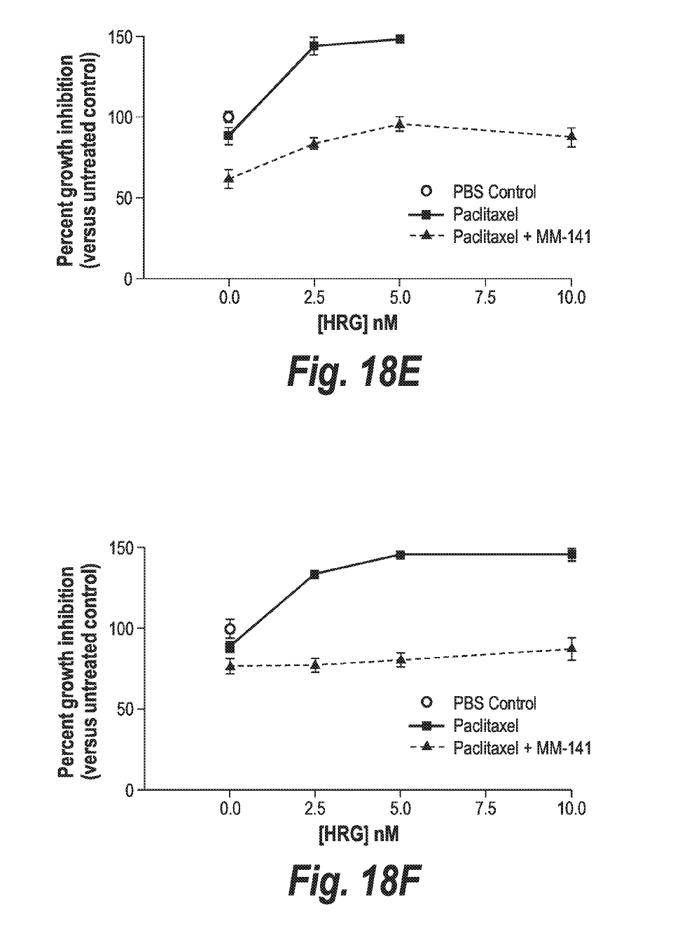

Preclinical experiments conducted with MM-141 have demonstrated the advantages of combining this regimen with paclitaxel. Administration of IGF-1 or heregulin abrogated the cytotoxic effect of paclitaxel on three-dimensional cultures of platinum-sensitive (Peol, PEA1 and OvCAR5) and platinum-resistant (Peo4 and PEA2) ovarian cancer cells in vitro and this resistance was reversed by addition of MM-141 (FIGS. 17A-B). Treatment of ovarian cancer cells with IGF-1 and/or HRG led to upregulation in AKT and ERK survival signaling, which could be abrogated by treatment with MM-141 (FIGS. 18A-F).

The cell viability assay (FIGS. 17A-B) was carried out using a CellTiter-Glo® (CTG) assay (Promega), which determines the number of viable cells in a culture based on quantitation of the ATP present. In FIGS. 17A-B, Peol, Peo4, PEA1, PEA2 and OvCAR5 cells were grown in 2% serum alone or with exogenous IGF-1 or HRG added (0-50 nM). Cell proliferation was measured following treatment with various concentrations of both ligands in the presence of paclitaxel (10-100 nM), either alone or in combination with MM-141 (1 μM).

In FIGS. 18A-F, Peol, Peo4, Ov90, PEA1, PEA2 and OvCAR8 ovarian cancer cells were plated on 10 cm plates (1-3×10⁶ cells per plate) in 10% serum-containing medium and incubated overnight. Once the cell density had reached approximately 70%, MM-141 (1 μM) was added for 1 hour, followed by addition of IGF-1 (50 nM) or HRG (10 nM) for 10 minutes, where indicated. Following treatment, cells were harvested in lysis buffer containing protease and phosphatase inhibitors and analyzed by western blotting. Phosphorylated and total levels of IGF-1R, ErbB3, AKT and ERK were determined using target-specific antibodies.

### Example 18

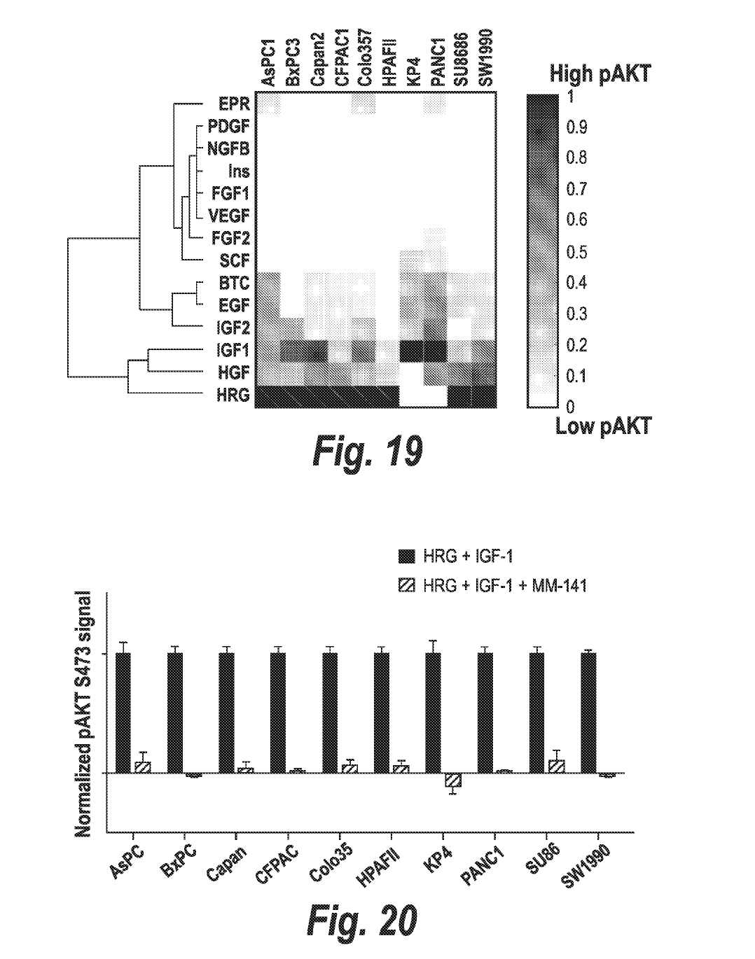

This Example discloses the effect of ligand stimulation on AKT activation in a panel of pancreatic cancer cell lines. Ten pancreatic cancer cell lines were separately seeded at 65% confluence in 10% serum-containing medium and incubated in 96-well plates overnight. The following day, medium on the cells was replaced with 2% serum-containing medium and cells were incubated for a further 24 hours. Following incubation, cells were treated with one of 14 different ligands at 100 ng/mL, or with PBS (control) for 15 minutes. After treatment, cells were harvested and protein lysates generated. Changes in pAKT levels across all treatments and cell lines were evaluated by pAKT ELISA. Phosphorylated AKT signal from untreated control was subtracted from each treatment, and then pAKT levels were max-normalized per cell line. Ligands and cell lines used are in Tables 2 and 3 below. Results are represented as a heat map in FIG. 19. The figure shows that AKT is activated by ErbB3 and IGF-1R ligands in a panel of pancreatic cancer cell lines.

TABLE 2
Ligands

| Ligand | Abbrev. | Cat # | Source |
|---|---|---|---|
| rh-Betacellulin | BTC | 100-50 | PreproTech |
| rh-Epidermal Growth Factor | EGF | AF-100-15 | PreproTech |
| rh-Epiregulin | EPR | 100-04 | PreproTech |
| rh-Fibroblast Growth Factor- | FGF-1 | 100-17A | PreproTech |
| acidic | | | |
| rh-Fibroblast Growth Factor- | FGF-2 | 100-18B | PreproTech |
| basic | | | |
| rh-Hepatocyte Growth Factor | HGF | 100-39 | PreproTech |
| rh-Heregulin-Beta-1 | HRG | 396-HB-050 | R&D Systems |
| rh-Insulin-like Growth Factor | IGF1 | 291-G1-200 | R&D Systems |
| 1 | | | |
| rh-Insulin-like Growth Factor | IGF2 | 100-12 | R&D Systems |
| 2 | | | |
| rh-Insulin | INS | I9278 | Sigma |
| rh-beta-Nerve Growth Factor | NGFB | 450-01 | PreproTech |
| rh-Platelet-Derived Growth | PDGF | 100-14B | PreproTech |
| Factor-BB | | | |
| rh-Stem Cell Factor | SCF | 300-07 | PreproTech |
| rh-Vascular Endothelial | VEGF | 100-20 | PreproTech |
| Growth Factor | | | |

(rh = recombinant human)

TABLE 3
Human Cell Lines

| Cell Line | Conditions |
|---|---|
| AsPC-1 | RPMI, P/S, 10% FBS |
| BxPC-3 | RPMI, P/S, 10% FBS |
| Capan-2 | McCoy's 5A, P/S, 10% FBS |
| CFPAC-1 | IMDM, P/S, 10% FBS |
| COLO 357 | RPMI, P/S, 10% FBS |
| HPAF-II | EMEM, P/S, 10% FBS |
| KP-4 | DMEM: F12, P/S, 10% FBS |
| PANC-1 | DMEM: F12, P/S, 10% FBS |
| SU.86.86 | RPMI, P/S, 10% FBS |
| SW 1990 | L-15, P/S, 10% FBS |

FBS = Fetal bovine serum

### Example 19

This Example shows that MM-141 blocks AKT activation induced by the combination of HRG and IGF-1. Ten pancreatic cancer cell lines (see Table 3 above) were seeded at 65% confluence in 10% serum-containing medium and incubated in 96-well plates overnight. The following day, medium on the cells was replaced with 2% serum-containing medium and cells were incubated for a further 24 hours with or without MM-141 (500 nM). Following incubation, cells were treated with a combination of HRG and IGF-1 ligands at 100 ng/mL or PBS (control) for 15 minutes. After treatment, cells were harvested and protein lysates generated. Changes in pAKT levels across all treatments and cell lines were evaluated by pAKT ELISA. Phosphorylated AKT signal from untreated control was subtracted from each treatment, and then pAKT levels post-treatment with HRG and IGF-1 were normalized to 1 for each cell line.

As shown in FIG. 20, ligand activated cells showed an increased production of pAKT, whereas cells that were incubated with MM-141 showed a greatly reduced amount of pAKT.

### Example 20

This Example shows that MM-141 potently down-regulates ErbB3 and IGF-1R in CFPAC-1 pancreatic cancer cells. CFPAC-1 pancreatic cancer cells were seeded at 65% confluence in 10% serum-containing medium and incubated in 96-well plates overnight. The following day, medium on the cells was replaced with 2% serum-containing medium for a further 24 hours, and following incubation, cells were treated with 50 nM of MM-141, ErbB3 mono-specific antibody (ErbB3 Ab) or IGF-1R mono-specific antibody (IGF-1R Ab), with PBS alone used as vehicle control. After treatment, cells were harvested and protein lysates generated. Changes in ErbB3 and IGF-1R levels across all treatments were evaluated by total ErbB3 or total IGF-1R ELISA, respectively.

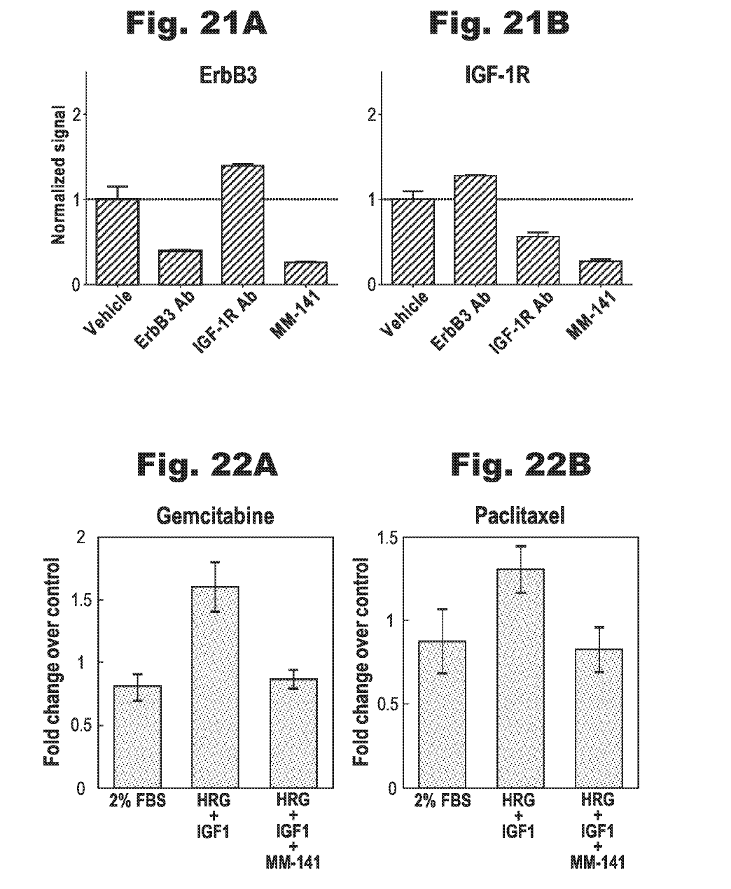

In FIGS. 21(A-B), bar graphs show (FIG. 21A) ErbB3 and (FIG. 21B) IGF-1R protein levels relative to vehicle control, which were normalized to 1.

### Example 21

This Example shows that HRG and IGF-1 render cells resistant to gemcitabine and paclitaxel and that MM-141 restores sensitivity to gemcitabine and paclitaxel in cells stimulated with HRG and IGF-1. CFPAC-1 pancreatic cancer cells were seeded in 10% serum-containing medium and incubated in 96-well three-dimensional nano-culture plates overnight. The following day, medium on the cells was replaced with 2% serum-containing medium for a further 24 hours, and following this incubation, cells were treated with gemcitabine (2 nM) or paclitaxel (6 nM), in the presence or absence of HRG (10 nM) and IGF-1 (50 nM), with or without MM-141 (1000 nM). Cell viability was measured 96 hours post-treatment using CellTiter-Glo® (Promega). As shown in FIGS. 22A-B, cells incubated with MM-141 in addition to the ligands had reduced proliferation compared to cells incubated with ligands alone. These data indicate that MM-141 resensistizes the cells to chemotherapy treatments.

### Example 22

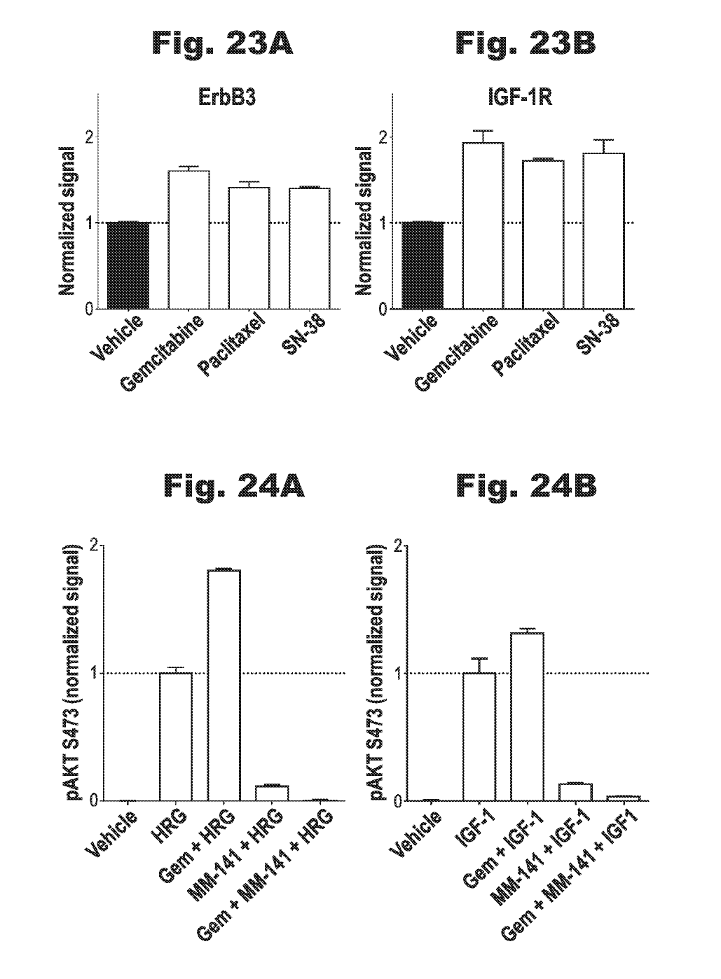

This Example shows that chemotherapy upregulates the receptors ErbB3 and IGF-1R. CFPAC-1 pancreatic cancer cells were seeded in 10% serum-containing medium in 10 cm plates for 3 days. Cells were treated with 1 μM gemcitabine, 1 μM paclitaxel, 1 μM SN-38, or PBS alone (vehicle) for 1 hour. After treatment, cells were harvested and protein lysates generated. Changes in (FIG. 23A) ErbB3 and (FIG. 23B) IGF-1R levels across all treatments were evaluated by the amount of total ErbB3 or total IGF-1R ELISA, respectively. FIGS. 23(A-B) are bar graphs showing protein levels relative to vehicle control, which were normalized to 1. These data indicate that pancreatic cancer cells develop resistance to chemotherapies by upregulating signaling receptors.

### Example 23

This Example shows that treatment with gemcitabine induces increased sensitivity to HRG and IGF-1, and that this effect is blocked by MM-141.

CFPAC-1 pancreatic cancer cells were seeded at 65% confluence in 10% serum-containing medium on 96-well plates overnight. The following day, medium on the cells was replaced with 2% serum-containing medium with or without MM-141 (500 nM), gemcitabine (1 μM) or the combination of MM-141 and gemcitabine (as dosed for the single agents) for 24 hours. Following incubation, cells were treated with (FIG. 24A) HRG (5 nM) or (FIG. 24B) IGF-1 (50 nM) for 15 minutes, where indicated. After treatment, cells were harvested and protein lysates generated. Changes in pAKT (Ser473) levels across all treatments were evaluated by pAKT ELISA, respectively. FIGS. 24(A-B) are bar graphs showing pAKT levels relative to ligand alone stimulated signals, which were normalized to 1.

### Example 24

This Example shows that paclitaxel induces increased sensitivity to IGF-1, and that this effect is blocked by MM-141.

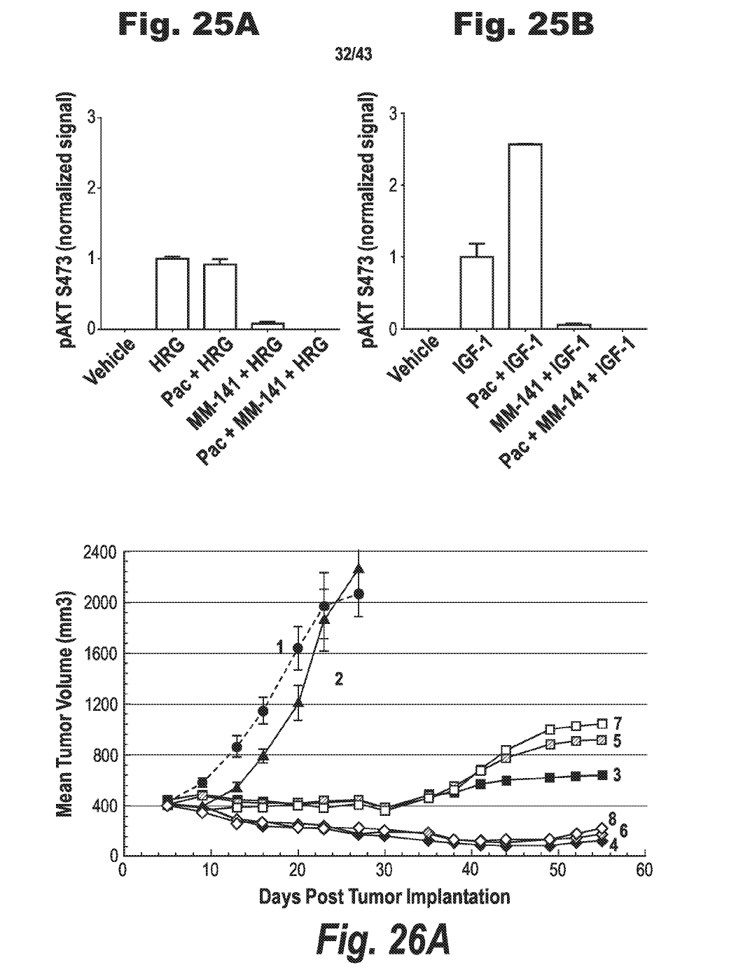

CFPAC-1 pancreatic cancer cells were seeded at 65% confluence in 10% serum-containing medium on 96-well plates overnight. The following day, medium on the cells was replaced with 2% serum-containing medium with or without MM-141 (500 nM), paclitaxel (100 nM) or the combination of MM-141 and gemcitabine as dosed for the single agents for 24 hours. Following incubation, cells were treated with (FIG. 25A) HRG (5 nM) or (FIG. 25B) IGF-1 (50 nM) for 15 minutes, where indicated. After treatment, cells were harvested and protein lysates generated. Changes in pAKT (Ser473) levels across all treatments were evaluated by pAKT ELISA, respectively. FIGS. 25(A-B) are bar graphs showing pAKT levels relative to ligand alone stimulated signals, which were normalized to 1.

### Example 25

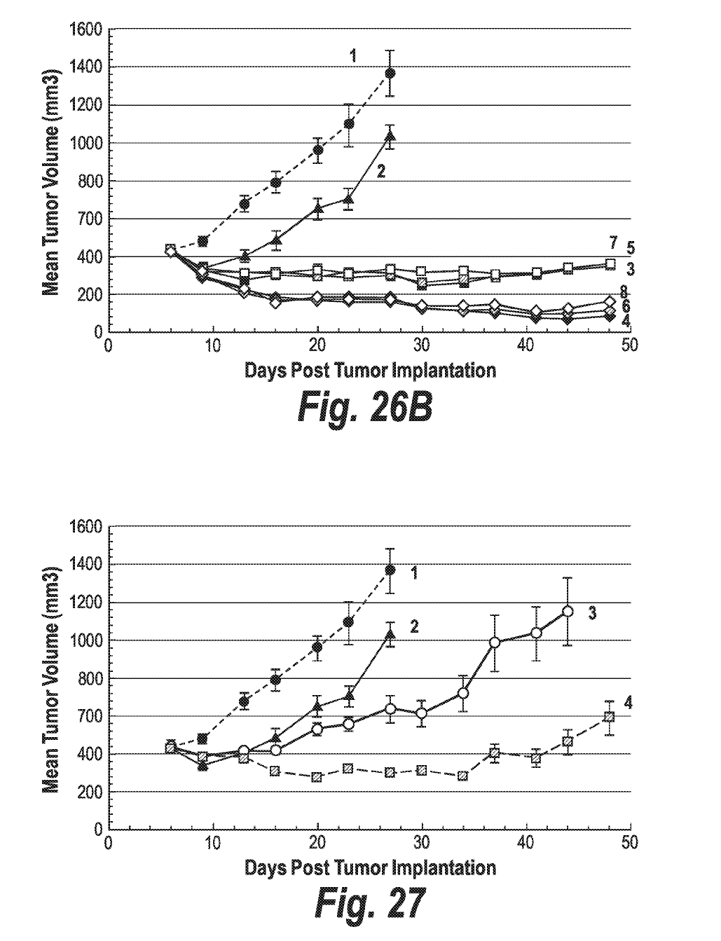

This Example discloses that MM-141 potentiates the effects of treatment with the combination of nab-paclitaxel and gemcitabine in vivo in HPAF-II (FIG. 26A) and CFPAC-1 (FIG. 26B) KRAS mutant pancreatic xenografts.

Results of this efficacy study are set forth in FIGS. 26(A-B). Tumors were established by inoculating female Fox Chase SCID-Beige mice subcutaneously with 5×10⁶ HPAF-II or CFPAC-1 cells, suspended 1:1 in 200 μL of Matrigel® Matrix Basement Membrane mix (Corning, Corning, N.Y.): unsupplemented culture media. When tumor volumes reached approximately 400 mm³, mice were randomized into study groups with equivalent average starting tumor volume per group maintained across all groups.

Mice were treated by i.p. injection with: (1) vehicle; (2) MM-141 (30 mg/kg, in PBS, q3d); (3) gemcitabine (20 mg/kg, in saline, q6d) and nab-paclitaxel (10 mg/kg, in saline, q3d); (4) the combination of MM-141 (30 mg/kg, in PBS, q3d) and gemcitabine (20 mg/kg, in saline, q6d) and nab-paclitaxel (10 mg/kg, in saline, q3d); (5) gemcitabine (10 mg/kg, in saline, q6d) and nab-paclitaxel (10 mg/kg, in saline, q3d); (6) MM-141 (30 mg/kg, in PBS, q3d) and gemcitabine (10 mg/kg, in saline, q6d) and nab-paclitaxel (10 mg/kg, in saline, q3d); (7) gemcitabine (5 mg/kg, in saline, q6d) and nab-paclitaxel (10 mg/kg, in saline, q3d) or (8) MM-141 (30 mg/kg, in PBS, q3d) and gemcitabine (5 mg/kg, in saline, q6d) and nab-paclitaxel (10 mg/kg, in saline, q3d).

### Example 26

This Example shows the effects of treatment with MM-141 and nab-paclitaxel, alone and in combination, on long-term growth on CFPAC-1 KRAS mutant pancreatic xenografts. Results of this efficacy study are set forth in FIG. 27. Tumors were established as described in Example 25. Mice were treated by i.p. injection with: (1) vehicle; (2) MM-141 (30 mg/kg, in PBS, q3d); (3) nab-paclitaxel (10 mg/kg, in saline, q3d); or (4) a combination of MM-141 (30 mg/kg, in PBS, q3d) and nab-paclitaxel (10 mg/kg, in saline, q3d).

As shown in FIG. 27, the combination of MM-141 and nab-paclitaxel showed the most inhibition of tumor growth as compared to either treatment alone.

### Example 27

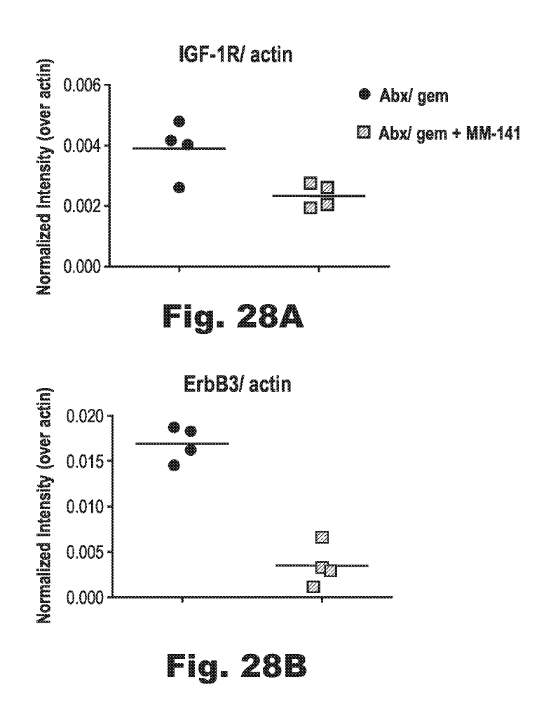

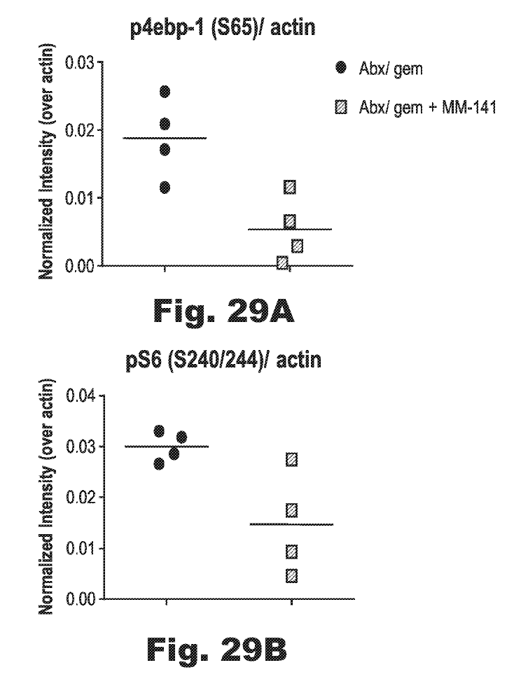

This Example shows the effect of treatment with nab-paclitaxel (Abx) and gemcitabine (gem), alone or in combination with MM-141, on membrane receptor levels in HPAF-II (FIGS. 28A-B) or CFPAC-1 (FIGS. 29A-B) xenograft tumors.

For the PD data shown in FIGS. 28(A-B) and 29(A-B), mice were treated with nab-paclitaxel (Abx), gemcitabine (gem)+/−MM-141 as described in Example 25. Tumors were harvested 24 hours after the final drug treatments, and then lysates were generated and subjected to western blotting analyses. Quantified immunoblot data are shown for (FIG. 28A) total IGF-1R, (FIG. 28B) total ErbB3, (FIG. 29A) phospho-4ebp-1 (S65), and (FIG. 29B) phospho-S6 (S240/244).

### Example 28

This Example shows the effect of nab-paclitaxel (Abx) and gemcitabine (gem) treatment, alone or in combination with MM-141, on intracellular signaling effector levels in CFPAC-1 xenografts.

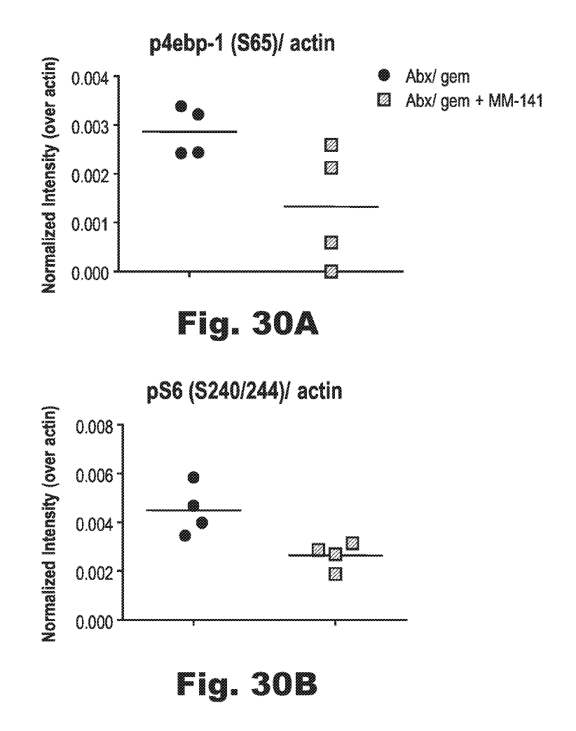

For the PD data shown in FIGS. 30(A-B), mice were treated with nab-paclitaxel (Abx), gemcitabine (gem)+/−MM-141 as described in Example 26. Tumors were harvested 24 hours after the final drug treatments; lysates were generated and subjected to western blotting analyses.

Quantified immunoblot data are shown for (FIG. 30A) phospho-4ebp-1 (S65) and (FIG. 30B) phospho-S6 (S240/244).

### Example 29

This Example demonstrates that MM-141 in a triple drug combination regimen induces sustained receptor down-regulation in an in vivo time course PD study in HPAF-II KRAS mutant pancreatic xenografts.

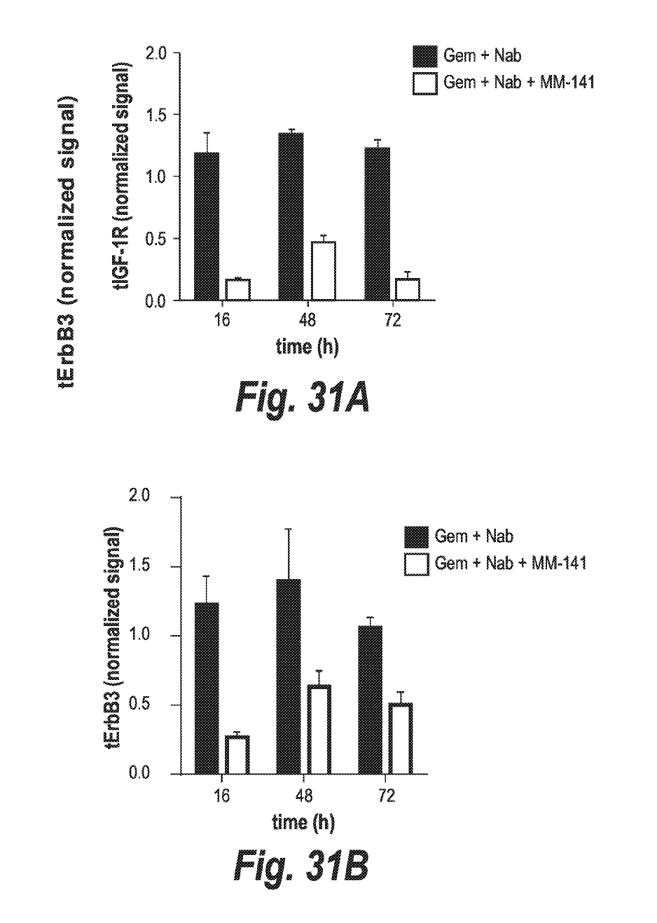

For the PD study results shown in FIGS. 31A-B, tumors were established by inoculating female Fox Chase SCID-Beige mice subcutaneously with 5×10⁶ HPAF-II cells, suspended 1:1 in 200 μL of Matrigel® Matrix Basement Membrane mix (Corning, Corning, N.Y.): unsupplemented culture media. When tumor volumes reached approximately 400 mm³, mice were randomized into study groups with equivalent average starting tumor volume per group maintained across all groups. Mice were treated by i.p. injection with: (1) nab-paclitaxel (Nab; 10 mg/kg, in saline) and gemcitabine (gem; 40 mg/kg, in saline); or (2) nab-paclitaxel and gemcitabine, and MM-141 (30 mg/kg, in PBS). Tumors were harvested at 16, 48 and 72 h after drug treatment, and lysates were generated and subjected to western blot analyses.

Quantified immunoblot data are shown for (FIG. 31A) total IGF-1R and (FIG. 31B) total ErbB3. As shown in the figures, xenograft tumor cells treated with the triple combination have a much lower membrane receptor level than xenograft tumor cells treated with the double combination of nab-paclitaxel and gemcitabine.

### Example 30

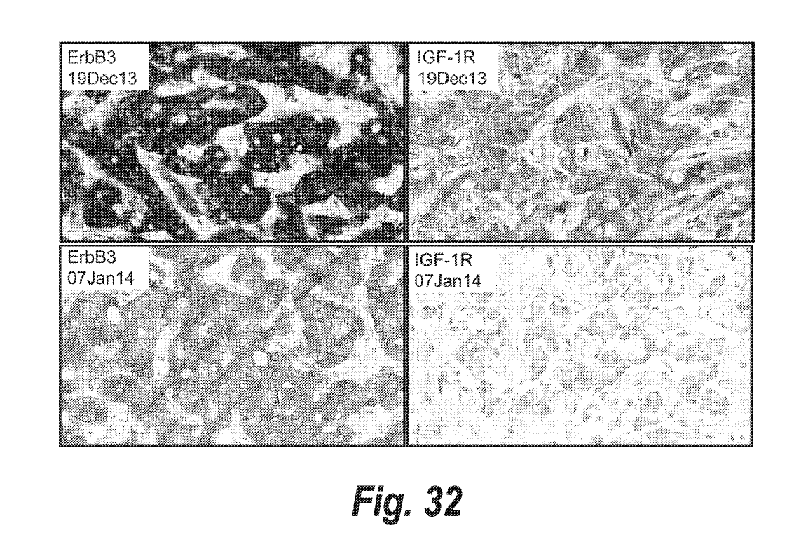

FIG. 32 shows the pre- (top panels) and post- (bottom panels) MM-141 treatment levels of ErbB3 (left panels) and IGF-1R (right panels), as detected by immunohistochemistry, in hepatocellular carcinoma tumor biopsies taken from a patient enrolled in an MM-141 Phase 1 clinical trial.

Formalin fixed, paraffin embedded biopsy samples were sectioned at 5 μm, processed and stained for IGF-1R (G11 clone detection antibody, Ventana) and ErbB3 (clone D22 antibody, Cell Signaling Technology). Down-regulation of both ErbB3 and IGF-1R following one month of MM-141 treatment is plainly evident in these (vertically) matched sections.

### Example 31

Treatment with MM-141 decreases the expression levels of IGF-1R and ErbB3 receptors to a greater extent than do individual monospecific antibodies targeting either IGF-1R or ErbB3.

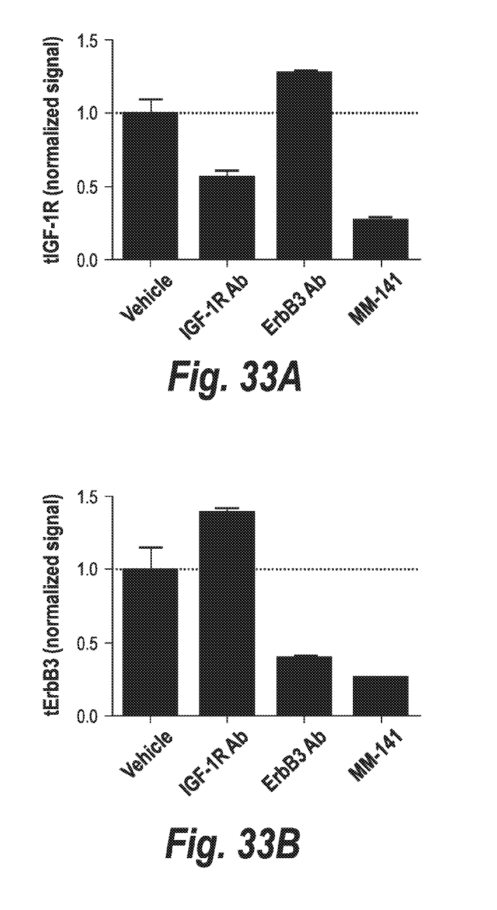

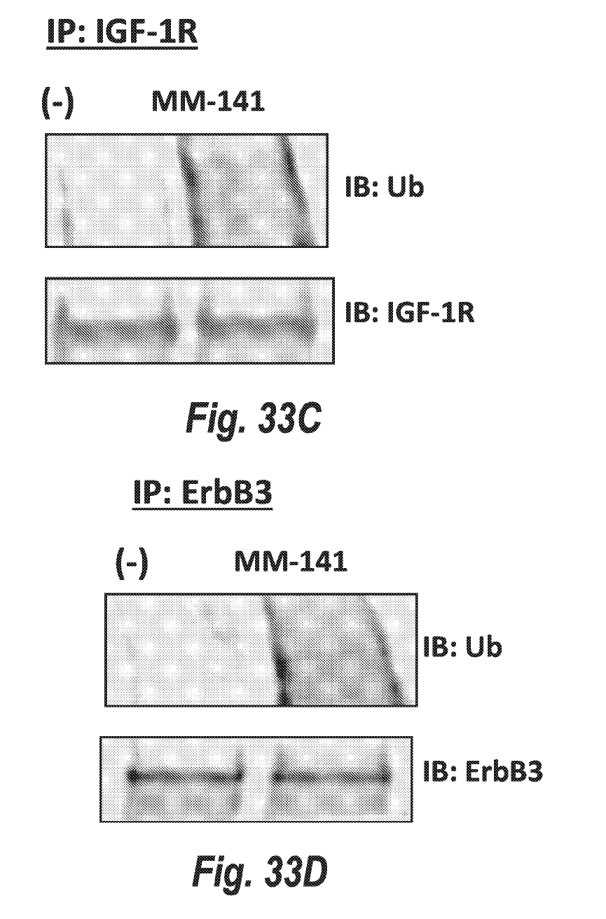

Cell lysates were harvested four hours post-treatment with antibodies as indicated in FIGS. 33A and 33B (50 nM of each antibody) and changes in receptor expression were measured by ELISA. All ELISA measurements are normalized to vehicle (PBS) treatment, and these measurements are expressed relative to a vehicle treated control value of 1.

In addition, the following experiments were performed, to investigate the mechanism of degradation of IGF-1R and ErbB3 receptors associated with MM-141 treatment.

Following 2 hours pre-treatment with 1 μM epoxomicin (a selective proteasome inhibitor), CFPAC-1 pancreatic cancer cells were treated with 500 nM MM-141 or vehicle for 20 minutes. Cell lysates were immunoprecipitated (IP) with an IGF-1R (FIG. 33C) or ErbB3 (FIG. 33D) antibody, and then immunoblotted (IB) for IGF-1R, ErbB3, or ubiquitin protein (Ub) expression by western blotting.

The results indicate that MM-141-induced IGF-1R and ErbB3 receptor downregulation is associated with induction of ubiquitination.

### Example 32

This Example shows that treatment of pancreatic cancer cells with gemcitabine induces increased expression of HRG.

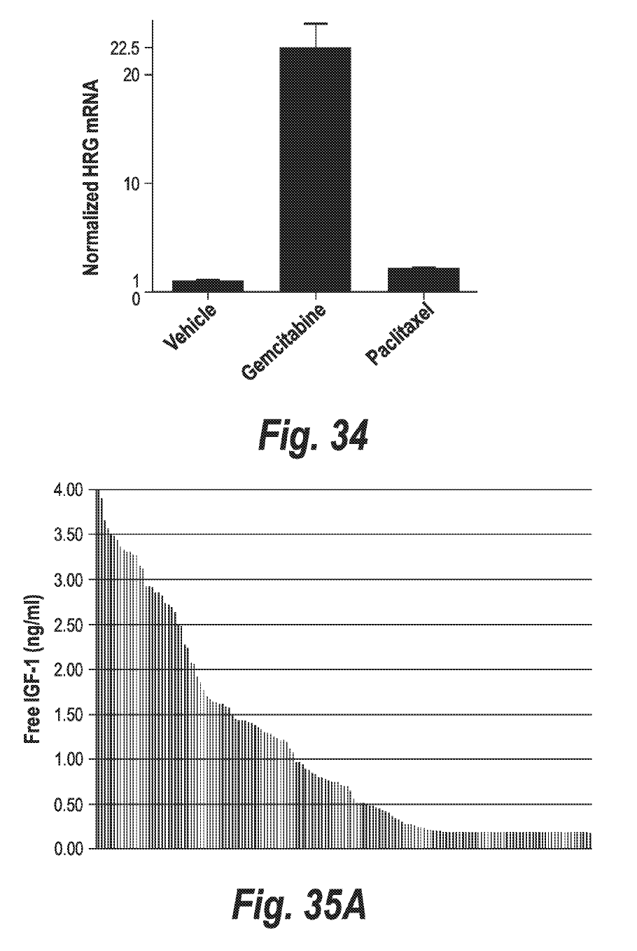

CFPAC-1 human pancreatic cancer cells were serum-starved by plating the cells in 2% serum-containing medium overnight, followed by treatment with vehicle, 500 nM gemcitabine, or 50 nM paclitaxel for 24 hours. Post-treatment, cell lysates were harvested, mRNA extracted, cDNA generated and changes in HRG mRNA expression evaluated by real time PCR. Bar graphs represent mean HRG mRNA expression relative to the geometric mean of three housekeeping genes (protein phosphatase 2 catalytic subunit alpha (PPP2CA), ribosomal protein L4 (RPL4), and glucuronidase beta (GUSB)), normalized to vehicle control. As shown in FIG. 34, treatment with paclitaxel at least doubles the amount of HRG mRNA relative to vehicle control, and treatment with gemcitabine increases it about 22-fold.

### Example 33

This Example shows that patients with high levels of free serum IGF-1 were able to remain on study longer than patients with lower levels of free serum IGF-1.

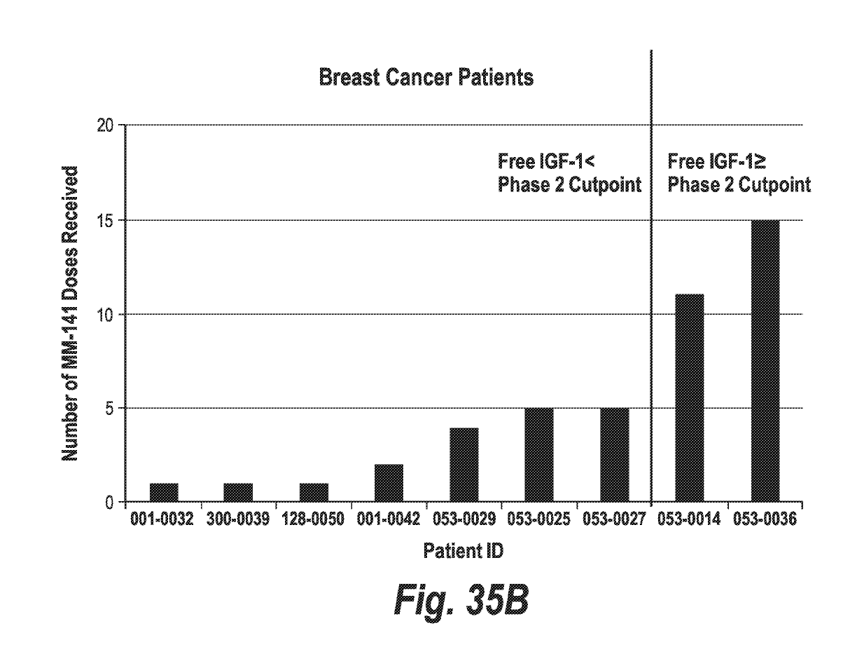

The assay used in this Example employs a novel receptor-capture based qualitative sandwich ELISA in the 96-well format. Free IGF-1 receptor is immobilized on each well of the microtiter plate. A series of standards, controls, and samples are pipetted into the wells and any free serum IGF-1 present is bound by the immobilized receptor. After washing away any unbound substances, a rabbit monoclonal antibody ((Cell Signaling Technology, Cat #9750)), specific for the anti-human IGF-1, is added to the wells, followed by another wash to remove any unbound substances. An enzyme-linked polyclonal anti-rabbit IgG HRP conjugate (Anti-Rabbit IgG, HRP-Linked antibody, Cell Signaling Technology, Catalog No. 7074) is added to the wells, followed by another wash to remove any unbound antibody-enzyme reagent. A 3,3′,5,5′-tetramethylbenzidine (TMB) substrate solution is added to the wells and color develops in proportion to the amount of Free IGF-1 bound in the initial step. The color development is stopped and the intensity of the color is measured. The optical density (OD) of each well of the ELISA plate is measured spectrophotometrically at a wavelength of 450 nm. A distribution of free serum IGF-1 in serum from pancreatic cancer patients is shown in FIG. 35A and summarized in Table 4 below. As the data in Table 4 show, median levels of free IGF-1 are higher in serum from Stage 3 pancreatic cancer patients, similar to what is seen in tissue samples from such patients. Approximately 60% of samples are expected to be HIGH (above cutpoint) regardless of the stage of cancer progression.

TABLE 4
Distribution of Free Serum IGF

| | Free IGF-1 (ng/ml) |
|---|---|
| Stage 3 (n = 101) | 0.80 |
| Stage 4 (n = 54) | 0.46 |
| All (n = 155) | 0.70 |
| | % Above Cutpoint |
| Stage 3 | 61% |
| Stage 4 | 56% |
| All | 59% |

Results:

Pre-treatment serum detection of free IGF-1 was seen in 5 of 7 (71.4%) patients who stayed on study long enough to receive more than two cycles of MM-141. These data support prospective selection of patients who have levels of free serum IGF-1 above 0.39 ng/mL to receive MM-141.

Retrospective analysis of the free IGF-1 found that in breast cancer patients, two patients with levels above the cutpoint remained on study longer and received at least twice the number of MM-141 doses as compared to those patients with levels below the cutpoint (FIG. 35B).

### Example 34

This Example discloses selection of a fixed-dose treatment regimen for MM-141.

To evaluate the difference between weight-based and fixed-dose regimens, a simulation study was conducted by comparing pharmacokinetics of these treatment options. Post-hoc estimates of PK parameters from each of the patients on the Phase 1 clinical study (see Example 15) were used in the simulation.

Population pharmacokinetic analyses of MM141 were performed based on pharmacokinetic data from patients treated with MM-141 monotherapy (n=13, 4 dose levels). The model was a two-compartmental model (ADVAN3) with covariate structure that includes relationship between weight-clearance and sex-clearance. Parameter estimates of the two-compartmental models and the associations were obtained from MM141 PK data. The inter-individual variabilities and residuals were assumed to be the same as those estimated from previously reported anti-ErbB3 antibody data; these assumed values were comparable to other antibodies. The residual followed a linear and proportional model. The simulation was performed by assuming a distribution of weight and sex as observed in patients in previously reported anti-ErbB3 antibody studies. The comparisons of dose regimens were controlled for inter-individual variability by applying multiple dose regimens for each simulated patient. The reported values were assumed to be at steady state. The models were as specified below.

CL=THETA(1)*EXP(ETA(1)+THETA(5)*(WEIGHT/MEDWGT−1)−THETA(6)*(SEX−1));

V1=THETA(2)*EXP(ETA(2))

Q=THETA(3)

V2=THETA(4)

where THETA(•) were fixed effect estimates, and ETA(•) were random effect estimates, MEDWGT was the median weight (=72), CL=clearance, V1=volume, Q=intercompartmental clearance, V2=volume of second compartment.

Preparation of dataset was performed in SAS (ENTERPRISE GUIDE 5.1) and R version 3.0.2. Population pharmacokinetic analysis was performed in NONMEM 7, using interface from PERL SPEAKS NONMEM (PSN). Post-NONMEM analysis was performed in R using XPOSE4 package version 4.5.0.

The simulation results showed comparable variability between both fixed-dosing and weight-based dosing regimens, suggesting there were no significant differences expected with a transition to a fixed-dosing schedule. Based on the model from patients treated on the monotherapy arm, a weight-based dosing of 40 mg/kg Q2W and a corresponding fixed dose of 2.8 grams Q2W had comparable maximum, minimum, average steady-state concentration levels, and variability; therefore 2.8 grams Q2W was the dose chosen for this Phase 2 study.

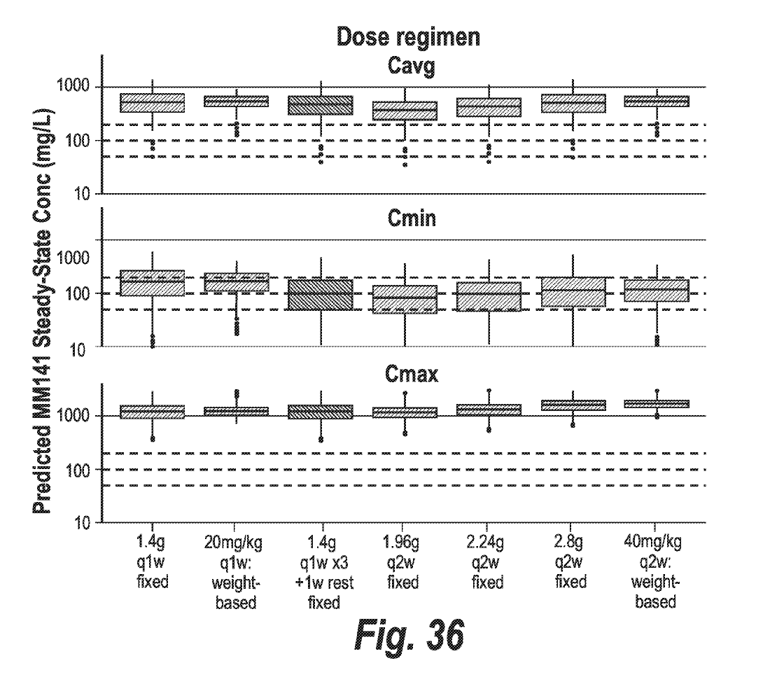

In order to evaluate a variety of MM-141 fixed-dosing options, a simulation study was conducted comparing the simulation pharmacokinetics (averaged and minimum concentration) of different dose concentrations (FIG. 36): 2.8 grams Q2W, 2.24 grams Q2W, 1.96 grams Q2W, 1.4 grams Q1W, 1.4 grams Q1W×3 with 1W off, 40 mg/kg Q2W, and 20 mg/kg Q1W. A dosing regimen of 2.8 grams Q2W is predicted to have a comparable imum concentration (Cmax) to 40 mg/kg Q2W, the highest dose level tested on the weight-based monotherapy dosing regimens. An alternative regimen of 2.24 grams Q2W is predicted to have a comparable Cmax to 20 mg/kg Q1W. The 2.24 grams Q2W dose had a predicted lower comparable minimum concentration (Cmin) to 20 mg/kg Q1W, but of the options tested, it was also predicted to provide the greatest number of patients with trough levels above 50 mg/L.

### EQUIVALENTS AND INCORPORATION BY REFERENCE

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure. The disclosure of each and every U.S. and foreign patent and pending patent application and publication referred to herein is specifically incorporated by reference herein in its entirety for all purposes.

1-167. (canceled)

168. A method of treating a patient with pancreatic cancer comprising administering to the patient a therapeutically effective amount of
(a) a bispecific binding molecule comprising:
i) an IGF-1R binding site comprising heavy and light chain variable regions, wherein the heavy chain variable region comprises a CDR1 comprising amino acid numbers 26-35 of SEQ ID NO: 3, a CDR2 comprising amino acid numbers 51-66 of SEQ ID NO: 3, a CDR3 comprising amino acid numbers 99-111 of SEQ ID NO: 3, and the light chain variable region comprises a CDR1 comprising amino acid numbers 24-34 of SEQ ID NO: 4, a CDR2 comprising amino acid numbers 50-56 of SEQ ID NO: 4, and a CDR3 comprising amino acid numbers 89-97 of SEQ ID NO: 4; and
ii) an ErbB3 binding site comprising heavy and light chain variable regions, wherein the heavy chain variable region comprises a CDR1 comprising amino acid numbers 492-501 of SEQ ID NO: 3, a CDR2 comprising amino acid numbers 517-532 of SEQ ID NO: 3, and a CDR3 comprising amino acid numbers 565-577 of SEQ ID NO: 3, and the light chain variable region comprises a CDR1 comprising amino acid numbers 634-644 of SEQ ID NO: 3, a CDR2 comprising amino acid numbers 660-666 of SEQ ID NO: 3, and a CDR3 comprising amino acid numbers 699-709 of SEQ ID NO: 3; and
(b) nab-paclitaxel.

169. The method of claim 168, wherein the patient is further administered gemcitabine.

170. The method of claim 168, wherein nab-paclitaxel is administered intravenously.

171. The method of claim 168, wherein the bispecific binding molecule is administered intravenously at a fixed dose of 2.8 grams every two weeks.

172. The method of claim 168, wherein nab-paclitaxel is administered at a dose of 125 mg/m² weekly.

173. The method of claim 169, wherein gemcitabine is administered at a dose of 1000 mg/m² weekly.

174. The method of claim 169, wherein nab-paclitaxel and gemcitabine are administered weekly for three weeks and followed by one week of rest.

175. A method of treating a patient with pancreatic cancer comprising administering to the patient a therapeutically effective amount of:
(a) a bispecific binding molecule comprising:
(i) an IGF-1R binding site comprising heavy and light chain variable regions, wherein the heavy chain variable region comprises amino acids 1-222 of SEQ ID NO: 3 and the light chain variable region comprises amino acids 1-107 of SEQ ID NO: 4, and
(ii) an ErbB3 binding site comprising heavy and light chain variable regions, wherein the heavy chain variable region the comprises amino acids 467-588 of SEQ ID NO: 3 and the light chain variable region comprises amino acids 612-720 of SEQ ID NO: 3; and
(b) nab-paclitaxel.

176. The method of claim 175, wherein the bispecific binding molecule comprises a heavy chain comprising the amino acid sequence of SEQ ID NO: 3, and a light chain comprising the amino acid sequence of SEQ ID NO: 4.

177. The method of claim 176, wherein the bispecific binding molecule is MM-141.

178. The method of claim 175, wherein the patient is further administered gemcitabine.

179. The method of claim 175, wherein nab-paclitaxel is administered intravenously.

180. The method of claim 177, wherein MM-141 is administered intravenously at a fixed dose of 2.8 grams every two weeks.

181. The method of claim 175, wherein nab-paclitaxel is administered at a dose of 125 mg/m² weekly.

182. The method of claim 178, wherein gemcitabine is administered at a dose of 1000 mg/m² weekly.

183. The method of claim 178, wherein nab-paclitaxel and gemcitabine are administered weekly for three weeks and followed by one week of rest.

184. A method for treating a patient having pancreatic cancer and identified as having a serum free IGF-1 concentration which is greater than about 15% below a median population level determined in a population having pancreatic cancer, the method comprising administering to the patient a therapeutically effective amount of a bispecific binding molecule comprising:
i) an IGF-1R binding site comprising heavy and light chain variable regions, wherein the heavy chain variable region comprises a CDR1 comprising amino acid numbers 26-35 of SEQ ID NO: 3, a CDR2 comprising amino acid numbers 51-66 of SEQ ID NO: 3, a CDR3 comprising amino acid numbers 99-111 of SEQ ID NO: 3, and the light chain variable region comprises a CDR1 comprising amino acid numbers 24-34 of SEQ ID NO: 4, a CDR2 comprising amino acid numbers 50-56 of SEQ ID NO: 4, and a CDR3 comprising amino acid numbers 89-97 of SEQ ID NO: 4; and
ii) an ErbB3 binding site comprising heavy and light chain variable regions, wherein the heavy chain variable region comprises a CDR1 comprising amino acid numbers 492-501 of SEQ ID NO: 3, a CDR2 comprising amino acid numbers 517-532 of SEQ ID NO: 3, and a CDR3 comprising amino acid numbers 565-577 of SEQ ID NO: 3, and the light chain variable region comprises a CDR1 comprising amino acid numbers 634-644 of SEQ ID NO: 3, a CDR2 comprising amino acid numbers 660-666 of SEQ ID NO: 3, and a CDR3 comprising amino acid numbers 699-709 of SEQ ID NO: 3.

185. The method of claim 184, wherein the patient has been identified as having a serum free IGF-1 concentration higher than the median population level.

186. The method of claim 184, wherein the patient has been identified as having a serum free IGF-1 concentration greater than about 5% below the median population level.

187. The method of claim 184, wherein the patient has been identified as having a serum free IGF-1 concentration greater than about 10% below the median population level.

188. A method for treating a patient having pancreatic cancer and identified as having a serum free IGF-1 concentration which is greater than about 15% below a median population level determined in a population having pancreatic cancer, the method comprising administering to the patient a therapeutically effective amount of a bispecific binding molecule comprising:
i) an IGF-1R binding site comprising heavy and light chain variable regions, wherein the heavy chain variable region comprises amino acids 1-222 of SEQ ID NO: 3 and the light chain variable region comprises amino acids 1-107 of SEQ ID NO: 4, and
ii) an ErbB3 binding site comprising heavy and light chain variable regions, wherein the heavy chain variable region comprises amino acids 467-588 of SEQ ID NO: 3 and the light chain variable region comprises amino acids 612-720 of SEQ ID NO: 3.

189. The method of claim 188, wherein the bispecific binding molecule comprises a heavy chain comprising the amino acid sequence of SEQ ID NO: 3, and a light chain comprising the amino acid sequence of SEQ ID NO: 4.

190. The method of claim 189, wherein the bispecific binding molecule is MM-141.

191. The method of claim 188, wherein the patient has been identified as having a serum free IGF-1 concentration higher than the median population level.

192. The method of claim 188, wherein the patient has been identified as having a serum free IGF-1 concentration greater than about 5% below the median population level.

193. The method of claim 188, wherein the patient has been identified as having a serum free IGF-1 concentration greater than about 10% below the median population level.

194. A method of treating a patient with pancreatic cancer comprising administering to the patient MM-141 intravenously at a fixed dose of 2.8 grams every two weeks.

195. The method of claim 194, further comprising administering to the patient nab-paclitaxel and gemcitabine as a four-week treatment cycle, wherein the nab-paclitaxel and gemcitabine are administered in each cycle weekly for three weeks followed by one week of rest.

196. The method of claim 195, wherein the nab-paclitaxel is administered at a dose of 125 mg/m² and the gemcitabine is administered at a dose of 1000 mg/m².
